SEQUENCE LISTING (191 sequences)

SEQ ID NO: 1 - DNA, 7430 bp, Homo sapiens
ggcggggcggcgcggcccgccaccatggagccccagcgccgggagctgctcgcccagtgtcagcagagcctggcccaggccatgacggaggtggaagccgtgctcgggctgctcgaggccgcgggagcgctcagtcccggcgagcggcggcagctggacgaggaggcgggaggcgccaaggcggagctgctgctcaagctgctcttggccaaggagcgggaccacttccaggacctgcgggcggcgctggagaagacgcagcctcacctgctgcccattctctacctgaacggcgtcgtcgggccgccgcagcccgccgaaggcgcgggttctacctacagcgtcctgtccaccatgccctcagactcagaaagcagcagctccctcagcagtgtgggcactaccgggaaggcgccgtccccaccacccctcctcactgaccagcaagtgaatgagaaggtggagaacctctccattcagctgcggctgatgacccgggagagaaacgagctccgcaagcgcctggcctttgctacgcatggcacggcctttgacaagaggccctaccacaggctgaatcctgactatgagaggctgaagatccagtgcgtgcgagccatgtcggacctgcagagcctgcagaaccagcacaccaacgccttgaagaggtgtgaggaggtggccaaggagactgacttctaccacacactccacagccggctcctgagtgaccagactcggctgaaggatgacgtggacatgctgaggcgggagaatgggcagctgctgcgggagcgaaacctgctgcagcagtcatgggaggacatgaagcggctccacgaggaggaccagaaggagatcggtgacctccgtgcccagcagcagcaggtgttgaagcacaacgggtcatccgagattctcaacaaactgtatgacacggccatggacaagttggaggtggtcaagaaggactatgacgcccttcggaagaggtacagtgagaaagtcgccatccacaatgcagacctgagccgcctggagcagctgggggaggagaaccagcggttgctgaagcagacagagatgctgacccagcagagggacacggccatccagctgcagcaccagtgcgccctctccctgaggaggtttgaggcgatccaccatgagctgaacaaggccacggcgcagaacaaggacctgcagtgggagatggagctgctgcagtcagagctgaccgagctgagaaccacgcaggtgaagacagcaaaggagtcggagaaatacagggaggagcgggacgctgtgtacagcgagtacaagctcatcatgagtgagcgtgaccaggtcatctctgagctggacaagctgcagaccgaagtggagctggccgagtccaagctcaagagcagcacatctgagaagaaggcggccaatgaggagatggaggcgctgcggcagatcaaagacacggtgacaatggatgctgggagagccaacaaggaggttgaaatccttcgaaagcagtgcaaggctctgtgccaggagctgaaggaagccctccaggaggcggatgtggccaagtgccggcgggactgggccttccaggagcgagacaagattgtagcagagcgtgacagcatccggacactgtgtgacaacctgaggcgggagcgggaccgtgcggtgagcgagctggctgaggccctgcgcagcctggatgacacccgcaagcagaagaatgatgtcagccgcgagctgaaggagctcaaggaacagatggaatcccagttggaaaaggaggcccggttccgacagctgatggcccacagctcccacgactcggccattgacacggattccatggagtgggaaacggaagttgtagagttcgagagggagacggaggatattgacttgaaggcactggggtttgatatggcagaaggtgtgaatgagccttgtttcccgggggactgtggcatatttgtcactaaagtggacaaaggaagcattgctgatggccgcttaagggtcaatgactggctgctgagaatcaacgatgtggacctcatcaacaaggacaagaagcaggccatcaaggcgctcctcaatggggagggggccatcaacatggtcgtgcggcggaggaagtccctgggtgggaaggtggtcacgccgctgcacatcaacctcagtggacagaaagacagtggcatcagtctggagaatggagtgtatgctgccgctgtgctgcctggaagccctgccgctaaagaagggtcccttgctgtgggagacaggatcgttgcgatcaatggcattgcactggacaacaagtctctgaatgaatgtgaatctctgctgcggagctgccaggactccctgaccctgtccctcctgaaggtattccctcagagctcctcgtggagtggccagaacatttttgaaaatatcaaagactctgataagatgctgagttttcgagcccatggcccggaggtccaggctcataacaaacggaacttgatacagcacaataactccacgcagacagacatcttctacacggacaggctggaagacaggaaggagccaggccccccaggaggcagcagctcctttctgcataagccattccctgggggacccttgcaggtctgcccccaggcctgtcccagtgcctctgagcgtagcctgagctccttccgctcagatgcctctggggaccgtggctttgggctggtggacgtgcgtggccggcggccactgctgccctttgagaccgaggtgggcccctgtggggttggggaggcctccctggacaaggcagactctgaaggctccaacagcggcgggacctggcccaaggccatgctcagctccacggcagtgcctgagaagctctctgtttataaaaagccaaagcaaagaaagtccatctttgaccctaacactttcaaacgcccccagacaccccccaaaatagactacctgcttccaggtcctgggcctgctcactctccccagccctccaagagggcggggcctctgacacccccaaaacctcccagaaggagcgactccattaagttccagcacaggctggagactagctccgagtcagaagccactctggtgggcagctccccatccactagtcccccgagcgccctgccccctgacgtggaccccggggagcccatgcacgcatcaccccctcgcaaggccagggtccgcattgcttccagctactaccctgaaggagatggggactcctcccacctgccggccaagaaatcctgtgatgaggacctcacctcccagaaggtggatgagctggggcagaagcgtcgccggccaaaatctgctcccagttttcggccgaagcttgctccagtagtgattcctgctcagttcctggaggaacagaagtgtgtcccggccagtggagaactctccccggagctccaggagtgggcaccttactcgcctgggcattccagccggcacagcaaccccccgctataccctagcaggccgtctgtgggcactgttccccggagtttgacccccagcaccactgtgagctccatcctgcggaaccccatctacactgtgcgcagtcacagggtcggcccctgcagctctccacctgcggcccgagatgctggcccccagggtttgcatcccagtgtccagcaccagggacgcctgagcctggacctgagccacaggacctgcagcgactactccgagatgagagccacccatgggtccaactcactgccctccagcgcccgcctgggttcttcgagtaacttgcagttcaaggcggaacgcattaaaatcccatcaacaccaagatatccgcggagtgtcgtgggctccgagagaggttcagtgtcacattctgaatgcagcactcctccacagtcacccctgaacatcgacaccctgtcctcttgtagccagtcccagacctcagcctccacattgcccagaatcgctgtcaaccccgcgtccctcggggagcggagaaaggacaggccttatgtggaggagccacgccacgtgaaggtgcagaagggctcagagccgctgggcatctccatcgtgagtggagagaagggcggcatctacgtctccaaggtgaccgtggggagcatcgctcaccaggctggcctcgagtatggggatcagttactggagttcaacggcataaacctgcggagcgccacggagcagcaggcgcggctcatcatcgggcagcagtgtgataccatcaccatcctggcccagtacaacccccacgtgcaccagctcagcagccactcccggtccagctcacacctggaccctgccggtacccactccactctccagggcagtggcaccaccaccccggagcatccatctgtcatcgacccactgatggagcaggacgaggggcctagcacccccccagccaagcagagcagctccaggattgcgggagatgccaacaagaagaccctggagccacgcgttgtcttcatcaaaaagtcccagctggagcttggggtgcacttgtgtggtgggaacctgcatggggtgtttgtggccgaggtggaggatgacagtcctgccaagggtcctgacggcctcgtgccaggggacctcatcctggagtatggcagcctggacgtgcggaacaagacagtggaggaagtctatgtggagatgctgaagcccagggatggcgtccgcctgaaggtgcagtaccgccctgaggagttcacgaaggccaagggcctgcctggtgacagcttctacatcagggccctgtacgaccggctggcagatgtggagcaagagttgagctttaagaaggacgacatcctctacgtggatgacaccttaccccagggcacgttcgggtcctggatggcttggcagctggacgagaatgcccagaagatccagcgcgggcagattcccagcaaatatgtgatggaccaagaattctccaggaggctcagcatgtctgaagtcaaagatgacaatagcgccacaaagacgctgtcagcggctgcacgccggtccttttttcggaggaaacacaagcacaaacgcagcgggtccaaggacgggaaagacctgctcgccttggatgccttttccagtgactccattccactctttgaagattcggtgagcctggcctatcagcgggtccagaaggtggactgcaccgctctgaggcctgtcctgattctggggcctttgctggacgtggtgaaggagatgctggtgaatgaggctcctggcaagttctgcagatgtccccttgaggtgatgaaggcctcccagcaggccattgagcggggtgtcaaagattgcctgtttgtcgactataagcggagaagcggccatttcgatgtgaccactgtggcgtcaataaaggagatcacagaaaagaaccgacactgcctcctggacattgctccgcacgctattgagcggctccaccacatgcacatctaccccattgtcatcttcatccactacaagagcgccaagcacatcaaggagcagagagaccccatctacctgagggacaaggtgactcagaggcattccaaagagcagtttgaggcggcgcagaagcttgagcaggagtacagcaggtacttcacaggggtcatccagggaggagccctgtcaagcatttgcactcagatcttggcaatggtcaatcaagaacaaaataaagtcctgtggattccagcctgcccgctctaggagaatgctgtgctgtggatgactgcagctggccgcctgaggggacaccagactcagctcttttctagcgactgaaagtagaagtctgtccgtctatgaacatgcgggggaaggatccggaaccaggacccagaagcacctcctttgtagacagagggccacggctgcgtgcgatccaggcccaggcccacacactctgcccgtgtcacacgtgtgctttaacacaaaacagataacactaaagacgggttcagcacccacctttctttagccagctgatcagagatgctgcaaagagaacctttcggatcactcgtttacaagccttttctaagtatttggtggtttatgtttacttgaacggctccatgttgccggtgcccagcccctgtcccctctgtcaaccccctgtcgctttggtgttggtttcgttcccgtcttcagcaaaacgaccttggaacctcaatgggggctgctttgctttgggaggttcttgttggtgggaccagagctttgacaaacctcctgctccttggtggcacctctcctggaaggacgtcacaactccaggtgctcagactgcctgtggcagcagaaccagtgcctttggcattttcctcccacaatggggaaggtgactttggcattcttacaaactcgtctctcggcctttctctcctgccttccacagcctctcgtttctcctccatctgtgcttattacttgaggactgtgtctgctccgtgagagctgcgtgggcagggctgcagtggggtccaggtggtgttcagctgtgctgatgcctgccattgggtcctccttaggctctgtaagtcgtgacagccttcatcagtgcaatgtttgcagggtaattcttaaactttttagagggtggcaggtacatcagttctttttgatatgaaaacattcatgtttcagacattgaattgagagcttttaggggaagcataatggttattgtcactatcaacagtctaaaaagaaaaactgaggtctttttaatcttgattacagcactcacggcatgcaccctactcagtgtgggtgtcttcgtttgggggctttttttttttttgcacttctgaggctagatatgtctggctgaagatttgatgtggttcctccttaagctatgcgtcctgttaataataggtactgtactgggctctgtgtaagtgtcgttggggtaggacctatattttaatactgttcctaacatttcattttactagcgagaaatctttgatttcattttattctttgtaattctagacactagattgtagtttagccataactgatgttttttaaaaagggatatattttcttgcacagttgttcaaaaaagagacaagtttcagtcctcaatgctgtcctttgttttacaggtacaagttttctagctcagacaaactatgaaaaactgtagactattctcaaggtattaactcgcagaccctctgggggtaggggctgttttctaagttacaggcagagtgggactgagatggtacagtgtgcacagacaggtactgagctgacagactgggattttctgtactaaaatgttactttgtataaaagttaaacaggctttagtacaacaaataaaggtcaatttctgt

SEQ ID NO: 2 - protein, 1919 aa, Homo sapiens
MEPQRRELLAQCQQSLAQAMTEVEAVLGLLEAAGALSPGERRQLDEEAGGAKAELLLKLLLAKERDHFQDLRAALEKTQPHLLPILYLNGVVGPPQPAEGAGSTYSVLSTMPSDSESSSSLSSVGTTGKAPSPPPLLTDQQVNEKVENLSIQLRLMTRERNELRKRLAFATHGTAFDKRPYHRLNPDYERLKIQCVRAMSDLQSLQNQHTNALKRCEEVAKETDFYHTLHSRLLSDQTRLKDDVDMLRRENGQLLRERNLLQQSWEDMKRLHEEDQKEIGDLRAQQQQVLKHNGSSEILNKLYDTAMDKLEVVKKDYDALRKRYSEKVAIHNADLSRLEQLGEENQRLLKQTEMLTQQRDTAIQLQHQCALSLRRFEAIHHELNKATAQNKDLQWEMELLQSELTELRTTQVKTAKESEKYREERDAVYSEYKLIMSERDQVISELDKLQTEVELAESKLKSSTSEKKAANEEMEALRQIKDTVTMDAGRANKEVEILRKQCKALCQELKEALQEADVAKCRRDWAFQERDKIVAERDSIRTLCDNLRRERDRAVSELAEALRSLDDTRKQKNDVSRELKELKEQMESQLEKEARFRQLMAHSSHDSAIDTDSMEWETEVVEFERETEDIDLKALGFDMAEGVNEPCFPGDCGIFVTKVDKGSIADGRLRVNDWLLRINDVDLINKDKKQAIKALLNGEGAINMVVRRRKSLGGKVVTPLHINLSGQKDSGISLENGVYAAAVLPGSPAAKEGSLAVGDRIVAINGIALDNKSLNECESLLRSCQDSLTLSLLKVFPQSSSWSGQNIFENIKDSDKMLSFRAHGPEVQAHNKRNLIQHNNSTQTDIFYTDRLEDRKEPGPPGGSSSFLHKPFPGGPLQVCPQACPSASERSLSSFRSDASGDRGFGLVDVRGRRPLLPFETEVGPCGVGEASLDKADSEGSNSGGTWPKAMLSSTAVPEKLSVYKKPKQRKSIFDPNTFKRPQTPPKIDYLLPGPGPAHSPQPSKRAGPLTPPKPPRRSDSIKFQHRLETSSESEATLVGSSPSTSPPSALPPDVDPGEPMHASPPRKARVRIASSYYPEGDGDSSHLPAKKSCDEDLTSQKVDELGQKRRRPKSAPSFRPKLAPVVIPAQFLEEQKCVPASGELSPELQEWAPYSPGHSSRHSNPPLYPSRPSVGTVPRSLTPSTTVSSILRNPIYTVRSHRVGPCSSPPAARDAGPQGLHPSVQHQGRLSLDLSHRTCSDYSEMRATHGSNSLPSSARLGSSSNLQFKAERIKIPSTPRYPRSVVGSERGSVSHSECSTPPQSPLNIDTLSSCSQSQTSASTLPRIAVNPASLGERRKDRPYVEEPRHVKVQKGSEPLGISIVSGEKGGIYVSKVTVGSIAHQAGLEYGDQLLEFNGINLRSATEQQARLIIGQQCDTITILAQYNPHVHQLSSHSRSSSHLDPAGTHSTLQGSGTTTPEHPSVIDPLMEQDEGPSTPPAKQSSSRIAGDANKKTLEPRVVFIKKSQLELGVHLCGGNLHGVFVAEVEDDSPAKGPDGLVPGDLILEYGSLDVRNKTVEEVYVEMLKPRDGVRLKVQYRPEEFTKAKGLPGDSFYIRALYDRLADVEQELSFKKDDILYVDDTLPQGTFGSWMAWQLDENAQKIQRGQIPSKYVMDQEFSRRLSMSEVKDDNSATKTLSAAARRSFFRRKHKHKRSGSKDGKDLLALDAFSSDSIPLFEDSVSLAYQRVQKVDCTALRPVLILGPLLDVVKEMLVNEAPGKFCRCPLEVMKASQQAIERGVKDCLFVDYKRRSGHFDVTTVASIKEITEKNRHCLLDIAPHAIERLHHMHIYPIVIFIHYKSAKHIKEQRDPIYLRDKVTQRHSKEQFEAAQKLEQEYSRYFTGVIQGGALSSICTQILAMVNQEQNKVLWIPACPL

SEQ ID NO: 3 - DNA, 166 bp, Homo sapiens
gaggcgggaggcgccaaggcggagctgctgctcaagctgctcttggccaaggagcgggaccacttccaggacctgcgggcggcgctggagaagacgcagcctcacctgctgcccattctctacctgaacggcgtcgtcgggccgccgcagcccgccgaaggcgcgg

SEQ ID NO: 4 - DNA, 93 bp, Homo sapiens
gtctcaacttaaactccagcaccacgaccctactactatctcgcacctgaaacaagctaacatgactaacacccttaattccatccaccctcc

SEQ ID NO: 5 - DNA, 544 bp, Homo sapiens
ggggctgtggccacgggggagcgggtggcggccgcggggccccggtgagccgggccggggtgcacgggggccgaggcggcctccgagccgggccggagctgtcgggagcctgggaaacacggcccaggcgactttctcccgggagttgtagtcttgctctgggggcggcgcgcggcccggctttcggggcgagttttctcgcggggcgggcggcgcgcgcggaggctgagggggagggggagcatgcccggctggagcccggcctcggggcgcgccccggccgccgtccccacctccgcctcctccttctcctcctccgtctcctcctcctcctcttccagtcccgccgccgcggcagccaacatggctgcgctccggagcccgggaggcggccgctgctgagcgccgcccgccgccccgggcccccagcccgcccaggccccggcccatggctcccgccgcctcacggctcccggcgccctagggctgctgccgaggtgctcgcctgcccgcccgccggcctcggggggcccgggaggcgtcc

SEQ ID NO: 6 - DNA, 31 bp, Homo sapiens
gctcggtggcagcgagtgagaggagctcagt

SEQ ID NO: 7 - DNA, 31 bp, Homo sapiens
gctcggtggcagcgaatgagaggagctcagt

SEQ ID NO: 8 - DNA, 31 bp, Homo sapiens
tggtctcccctctttccccaggttctaccta

SEQ ID NO: 9 - DNA, 31 bp, Homo sapiens
tggtctcccctcttttcccaggttctaccta

SEQ ID NO: 10 - DNA, 31 bp, Homo sapiens
gtgggtgagtaccaccgtctggggaggacac

SEQ ID NO: 11 - DNA, 31 bp, Homo sapiens
gtgggtgagtaccactgtctggggaggacac

SEQ ID NO: 12 - DNA, 31 bp, Homo sapiens
ccctcctcactgaccagcaagtgaatgagaa

SEQ ID NO: 13 - DNA, 31 bp, Homo sapiens
ccctcctcactgaccggcaagtgaatgagaa

SEQ ID NO: 14 - DNA, 31 bp, Homo sapiens
ccgcaagcgcctggcctttgctacgcatggc

SEQ ID NO: 15 - DNA, 31 bp, Homo sapiens
ccgcaagcgcctggcgtttgctacgcatggc

SEQ ID NO: 16 - DNA, 31 bp, Homo sapiens
ctgagtgtcccctttcccccacctcatgtcc

SEQ ID NO: 17 - DNA, 31 bp, Homo sapiens
ctgagtgtccccttttccccacctcatgtcc

SEQ ID NO: 18 - DNA, 31 bp, Homo sapiens
ttcagcacactccacagccggctcctgagtg

SEQ ID NO: 19 - DNA, 31 bp, Homo sapiens
ttcagcacactccacggccggctcctgagtg

SEQ ID NO: 20 - DNA, 31 bp, Homo sapiens
cccagcccctggagactggccatttctccca

SEQ ID NO: 21 - DNA, 31 bp, Homo sapiens
cccagcccctggagaatggccatttctccca

SEQ ID NO: 22 - DNA, 31 bp, Homo sapiens
ggcagccacccacctactcagatccagccta

SEQ ID NO: 23 - DNA, 31 bp, Homo sapiens
ggcagccacccacctcctcagatccagccta

SEQ ID NO: 24 - DNA, 31 bp, Homo sapiens
gagaaccacgcaggtgaagacagcaaaggag

SEQ ID NO: 25 - DNA, 31 bp, Homo sapiens
gagaaccacgcaggtaaagacagcaaaggag

SEQ ID NO: 26 - DNA, 31 bp, Homo sapiens
ctcagctgtggtggatagactggacagtgcc

SEQ ID NO: 27 - DNA, 31 bp, Homo sapiens
ctcagctgtggtggacagactggacagtgcc

SEQ ID NO: 28 - DNA, 31 bp, Homo sapiens
gaagttgtagagttcgagagggagacggtaa

SEQ ID NO: 29 - DNA, 31 bp, Homo sapiens
gaagttgtagagttccagagggagacggtaa

SEQ ID NO: 30 - DNA, 30 bp, Homo sapiens
ggagtgtatgctgccgctgtgctgcctgga

SEQ ID NO: 31 - DNA, 30 bp, Homo sapiens
ggagtgtatgctgctgctgtgctgcctgga

SEQ ID NO: 32 - DNA, 37 bp, Homo sapiens
gcggtaagtctcaaggctggagccagggtcatctgcc

SEQ ID NO: 33 - DNA, 30 bp, Homo sapiens
gcggtaagtctcaagccagggtcatctgcc

SEQ ID NO: 34 - DNA, 31 bp, Homo sapiens
ggtaggcctgaggccgctctgcctgtggcct

SEQ ID NO: 35 - DNA, 31 bp, Homo sapiens
ggtaggcctgaggccactctgcctgtggcct

SEQ ID NO: 36 - DNA, 31 bp, Homo sapiens
tgaatctctgctgcgcagctgccaggactcc

SEQ ID NO: 37 - DNA, 31 bp, Homo sapiens
tgaatctctgctgcggagctgccaggactcc

SEQ ID NO: 38 - DNA, 31 bp, Homo sapiens
catgctactccttggggtcacaggatccttg

SEQ ID NO: 39 - DNA, 31 bp, Homo sapiens
catgctactccttggagtcacaggatccttg

SEQ ID NO: 40 - DNA, 31 bp, Homo sapiens
cggggagcccatgcacgcatcaccccctcgc

SEQ ID NO: 41 - DNA, 31 bp, Homo sapiens
cggggagcccatgcatgcatcaccccctcgc

SEQ ID NO: 42 - DNA, 31 bp, Homo sapiens
acgcatcaccccctcgcaaggccagggtccg

SEQ ID NO: 43 - DNA, 31 bp, Homo sapiens
acgcatcaccccctcacaaggccagggtccg

SEQ ID NO: 44 - DNA, 31 bp, Homo sapiens
actcctcccacctgccggccaagaaatcctg

SEQ ID NO: 45 - DNA, 31 bp, Homo sapiens
actcctcccacctgctggccaagaaatcctg

SEQ ID NO: 46 - DNA, 31 bp, Homo sapiens
ctgggtcctttggggcgtcttttctcaccaa

SEQ ID NO: 47 - DNA, 31 bp, Homo sapiens
ctgggtcctttggggtgtcttttctcaccaa

SEQ ID NO: 48 - DNA, 31 bp, Homo sapiens
aagcccatttctaggcactgttccccggagt

SEQ ID NO: 49 - DNA, 31 bp, Homo sapiens
aagcccatttctaggtactgttccccggagt

SEQ ID NO: 50 - DNA, 31 bp, Homo sapiens
tacgcttctctgtaccccagctgcccaagcc

SEQ ID NO: 51 - DNA, 31 bp, Homo sapiens
tacgcttctctgtactccagctgcccaagcc

SEQ ID NO: 52 - DNA, 31 bp, Homo sapiens
tcctgggactgagctgatttctctactggga

SEQ ID NO: 53 - DNA, 31 bp, Homo sapiens
tcctgggactgagctcatttctctactggga

SEQ ID NO: 54 - DNA, 31 bp, Homo sapiens
gagtgtcgtgggctccgagagaggtaaggac

SEQ ID NO: 55 - DNA, 31 bp, Homo sapiens
gagtgtcgtgggctctgagagaggtaaggac

SEQ ID NO: 56 - DNA, 31 bp, Homo sapiens
tcctagggaacagcagtgctcccaagtcccc

SEQ ID NO: 57 - DNA, 31 bp, Homo sapiens
tcctagggaacagcactgctcccaagtcccc

SEQ ID NO: 58 - DNA, 31 bp, Homo sapiens
acacctggaccctgccggtacccactccact

SEQ ID NO: 59 - DNA, 31 bp, Homo sapiens
acacctggaccctgctggtacccactccact

SEQ ID NO: 60 - DNA, 31 bp, Homo sapiens
ggcctagcacccccccagccaagcagagcag

SEQ ID NO: 61 - DNA, 31 bp, Homo sapiens
ggcctagcaccccccaagccaagcagagcag

SEQ ID NO: 62 - DNA, 31 bp, Homo sapiens
aggagggatgttgaatttctgccgtatggtc

SEQ ID NO: 63 - DNA, 31 bp, Homo sapiens
aggagggatgttgaaattctgccgtatggtc

SEQ ID NO: 64 - DNA, 31 bp, Homo sapiens
gccgtatggtcagcactggcccctctcgggt

SEQ ID NO: 65 - DNA, 31 bp, Homo sapiens
gccgtatggtcagcagtggcccctctcgggt

SEQ ID NO: 66 - DNA, 31 bp, Homo sapiens
gcactggcccctctcgggtgcccaagctgcc

SEQ ID NO: 67 - DNA, 31 bp, Homo sapiens
gcactggcccctctcaggtgcccaagctgcc

SEQ ID NO: 68 - DNA, 31 bp, Homo sapiens
gagctttaagaaggacgacatcctctacgtg

SEQ ID NO: 69 - DNA, 31 bp, Homo sapiens
gagctttaagaaggatgacatcctctacgtg

SEQ ID NO: 70 - DNA, 31 bp, Homo sapiens
ggggtggggtggggcaggggtcgccgagggc

SEQ ID NO: 71 - DNA, 30 bp, Homo sapiens
ggggtggggtggggcggggtcgccgagggc

SEQ ID NO: 72 - DNA, 31 bp, Homo sapiens
agggcagcagggtcttgatggccctgcccag

SEQ ID NO: 73 - DNA, 31 bp, Homo sapiens
agggcagcagggtctcgatggccctgcccag

SEQ ID NO: 74 - DNA, 31 bp, Homo sapiens
agatgacaatagcgccacaaagacgctgtca

SEQ ID NO: 75 - DNA, 31 bp, Homo sapiens
agatgacaatagcgctacaaagacgctgtca

SEQ ID NO: 76 - DNA, 31 bp, Homo sapiens
ggttactgacagctgctgagcagtgttcttc

SEQ ID NO: 77 - DNA, 31 bp, Homo sapiens
ggttactgacagctgatgagcagtgttcttc

SEQ ID NO: 78 - DNA, 31 bp, Homo sapiens
cctggatgcctgggacgacagacatgacaga

SEQ ID NO: 79 - DNA, 31 bp, Homo sapiens
cctggatgcctgggatgacagacatgacaga

SEQ ID NO: 80 - DNA, 31 bp, Homo sapiens
ggctgttttcttagccgtggagaagcccgcg

SEQ ID NO: 81 - DNA, 31 bp, Homo sapiens
ggctgttttcttagctgtggagaagcccgcg

SEQ ID NO: 82 - DNA, 31 bp, Homo sapiens
gccgcctgaggggacgccagactcagctctt

SEQ ID NO: 83 - DNA, 31 bp, Homo sapiens
gccgcctgaggggacaccagactcagctctt

SEQ ID NO: 84 - DNA, 31 bp, Homo sapiens
aagtagaagtctgtccgtctatgaacatgcg

SEQ ID NO: 85 - DNA, 31 bp, Homo sapiens
aagtagaagtctgtctgtctatgaacatgcg

SEQ ID NO: 86 - DNA, 31 bp, Homo sapiens
tgtctatgaacatgcaggggaaggatccgga

SEQ ID NO: 87 - DNA, 31 bp, Homo sapiens
tgtctatgaacatgcgggggaaggatccgga

SEQ ID NO: 88 - DNA, 31 bp, Homo sapiens
ctctcctggaaggacgtcacaactccaggtg

SEQ ID NO: 89 - DNA, 31 bp, Homo sapiens
ctctcctggaaggacatcacaactccaggtg

SEQ ID NO: 90 - DNA, 31 bp, Homo sapiens
tctccagaagcttcactcacactccactggt

SEQ ID NO: 91 - DNA, 31 bp, Homo sapiens
tctccagaagcttcagtcacactccactggt

SEQ ID NO: 92 - DNA, 31 bp, Homo sapiens; identical to SEQ ID NO: 91

SEQ ID NO: 93 - DNA, 31 bp, Homo sapiens
agagttagacttcttgaacaaccttttaagg

SEQ ID NO: 94 - DNA, 19 bp, Homo sapiens
ccatgacggaggtggaagc

SEQ ID NO: 95 - DNA, 19 bp, Homo sapiens
agaggagcgagtccaccga

SEQ ID NO: 96 - DNA, 20 bp, Homo sapiens
cggaaggatgatcctgtgag

SEQ ID NO: 97 - DNA, 16 bp, Homo sapiens
ccaggggaggatgcaa

SEQ ID NO: 98 - DNA, 21 bp, Homo sapiens
acaagcacaccactatcaggg

SEQ ID NO: 99 - DNA, 20 bp, Homo sapiens
cctaatccaggacctggttc

SEQ ID NO: 100 - DNA, 21 bp, Homo sapiens
cttgcacagggacaggactag

SEQ ID NO: 101 - DNA, 22 bp, Homo sapiens
gctgtatctacgggaagtgttg

SEQ ID NO: 102 - DNA, 21 bp, Homo Sapiens
gatcacagatgtgagccaacg

SEQ ID NO: 103 - DNA, 22 bp, Homo sapiens
ccttgtcatcagtctcaccctc

SEQ ID NO: 104 - DNA, 20 bp, Homo sapiens
gagccacgattcccaagaca

SEQ ID NO: 105 - DNA, 20 bp, Homo sapiens
acatctcgccacctctcttg

SEQ ID NO: 106 - DNA, 20 bp, Homo sapiens
tgctgtaggagaggctgaaa

SEQ ID NO: 107 - DNA, 20 bp, Homo sapiens
tcagcaacctctccctcttc

SEQ ID NO: 108 - DNA, 18 bp, Homo sapiens
acgccagcttgaggtcac

SEQ ID NO: 109 - DNA, 20 bp, Homo sapiens
tgaccttgtcctctgctcct

SEQ ID NO: 110 - DNA, 18 bp, Homo sapiens
cattgccttgcccagaag

SEQ ID NO: 111 - DNA, 20 bp, Homo sapiens
ccgtggctctctctgttcac

SEQ ID NO: 112 - DNA, 20 bp, Homo sapiens
tcaccgtctcctccttcatc

SEQ ID NO: 113 - DNA, 19 bp, Homo sapiens
tttgtgaaatggttgctgt

SEQ ID NO: 114 - DNA, 20 bp, Homo sapiens
gtgctacctggctctcttcg

SEQ ID NO: 115 - DNA, 20 bp, Homo sapiens
cccctgagtgagagttgtgg

SEQ ID NO: 116 - DNA, 20 bp, Homo sapiens
ccactgaggttgatgtgcag

SEQ ID NO: 117 - DNA, 22 bp, Homo sapiens
caccagggtagatgtaactgag

SEQ ID NO: 118 - DNA, 22 bp, Homo sapiens
gattcttatttccctcccagac

SEQ ID NO: 119 - DNA, 20 bp, Homo sapiens
ctcctgactttggcaccttg

SEQ ID NO: 120 - DNA, 20 bp, Homo sapiens
ttcagaccagcgtccagtca

SEQ ID NO: 121 - DNA, 20 bp, Homo sapiens
tgctttacctctggggatgg

SEQ ID NO: 122 - DNA, 20 bp, Homo sapiens
cttccgctcagatgcctctg

SEQ ID NO: 123 - DNA, 22 bp, Homo sapiens
cagaaggagcgactccattaag

SEQ ID NO: 124 - DNA, 20 bp, Homo sapiens
gcaaaggcaccaggctaaac

SEQ ID NO: 125 - DNA, 21 bp, Homo sapiens
ggtagtagctggaagcaatgc

SEQ ID NO: 126 - DNA, 21 bp, Homo sapiens
cagagagcttctcaggcactg

SEQ ID NO: 127 - DNA, 20 bp, Homo sapiens
tggccacactccactctttc

SEQ ID NO: 128 - DNA, 20 bp, Homo sapiens
ctcagggctgaaaacacatg

SEQ ID NO: 129 - DNA, 20 bp, Homo sapiens
gagccacagccacattgtga

SEQ ID NO: 130 - DNA, 20 bp, Homo sapiens
ggaagcttctccaccaatga

SEQ ID NO: 131 - DNA, 20 bp, Homo sapiens
gaacccttgcctggtctgtg

SEQ ID NO: 132 - DNA, 20 bp, Homo sapiens
gaaagcaatggctctgacag

SEQ ID NO: 133 - DNA, 20 bp, Homo sapiens
gactggtagcctggtggaga

SEQ ID NO: 134 - DNA, 21 bp, Homo sapiens
gaagttctcagctaagcccag

SEQ ID NO: 135 - DNA, 20 bp, Homo sapiens
gcaatgcagagcctagcatc

SEQ ID NO: 136 - DNA, 20 bp, Homo sapiens
tgctgggacactcaagctac

SEQ ID NO: 137 - DNA, 21 bp, Homo sapiens
ctgcactgtcagatcatatgc

SEQ ID NO: 138 - DNA, 20 bp, Homo sapiens
acaccaggatgggctcagtg

SEQ ID NO: 139 - DNA, 20 bp, Homo sapiens
gaacagcagtgctcccaagt

SEQ ID NO: 140 - DNA, 20 bp, Homo sapiens
ccagaacttacggctggcac

SEQ ID NO: 141 - DNA, 22 bp, Homo sapiens
gctcagatctagttgccacagg

SEQ ID NO: 142 - DNA, 21 bp, Homo sapiens
ccacttggagaatgtgctcag

SEQ ID NO: 143 - DNA, 20 bp, Homo sapiens
ccaagaagcaggcagaaagc

SEQ ID NO: 144 - DNA, 21 bp, Homo sapiens
tgtactcctccgtctttggtg

SEQ ID NO: 145 - DNA, 21 bp, Homo sapiens
gactcagtccttcctgcagag

SEQ ID NO: 146 - DNA, 21 bp, Homo sapiens
caccaggaaaagagtctccag

SEQ ID NO: 147 - DNA, 20 bp, Homo sapiens
ctcggcgattcctgatcaag

SEQ ID NO: 148 - DNA, 21 bp, Homo sapiens
gaagcagaatccctcctccag

SEQ ID NO: 149 - DNA, 21 bp, Homo sapiens
gtagccttgagacctgccaag

SEQ ID NO: 150 - DNA, 20 bp, Homo sapiens
tgtggctctgaagatggcag

SEQ ID NO: 151 - DNA, 20 bp, Homo sapiens
gtgctcatgctggactccag

SEQ ID NO: 152 - DNA, 20 bp, Homo sapiens
caggcttctggaacactgtg

SEQ ID NO: 153 - DNA, 20 bp, Homo sapiens
gtcagattcatgcatggcag

SEQ ID NO: 154 - DNA, 21 bp, Homo sapiens
caggcacaggtgaactcagac

SEQ ID NO: 155 - DNA, 21 bp, Homo sapiens
ctgtgtggctttactgccttg

SEQ ID NO: 156 - DNA, 21 bp, Homo sapiens
ccataggcccatctctcattc

SEQ ID NO: 157 - DNA, 21 bp, Homo sapiens
gctgttgctgtgctttatgtg

SEQ ID NO: 158 - DNA, 20 bp, Homo sapiens
agaatcctgacgttggccag

SEQ ID NO: 159 - DNA, 21 bp, Homo sapiens
ctggtgaaggagagtcaggtg

SEQ ID NO: 160 - DNA, 20 bp, Homo sapiens
gtgcttctgggtcctggttc

SEQ ID NO: 161 - DNA, 23 bp, Homo sapiens
ccagtgactccattccactcttt

SEQ ID NO: 162 - DNA, 19 bp, Homo sapiens
cggtgcagtccaccttctg

SEQ ID NO: 163 - DNA, 18 bp, Homo sapiens
ggagaagcggccatttcg

SEQ ID NO: 164 - DNA, 20 bp, Homo sapiens
ctcaatagcgtgcggagcaa

SEQ ID NO: 165 - DNA, 22 bp, Homo sapiens
cagtgtgggtgtcttcgtttgg

SEQ ID NO: 166 - DNA, 25 bp, Homo sapiens
attaacaggacgcatagcttaagga

SEQ ID NO: 167 - DNA, 31 bp, Homo sapiens
aaaggagatcacagaaaagaaccgacactgc

SEQ ID NO: 168 - DNA, 35 bp, Homo sapiens
tgaggctagatatgtctggctgaagatttgatgtg

SEQ ID NO: 169 - DNA, 24 bp, Homo sapiens
ccattctatcatcaacgggtacaa

SEQ ID NO: 170 - DNA, 22 bp, Homo sapiens
agcaagtgggaaggtgtaatcc

SEQ ID NO: 171 - DNA, 17 bp, Homo sapiens
agcctcgcctttgccga

SEQ ID NO: 172 - DNA, 15 bp, Homo sapiens
ctggtgcctggggcg

SEQ ID NO: 173 - DNA, 22 bp, Homo sapiens
ccgccgcccgtccacacccgcc

SEQ ID NO: 174 - DNA, 21 bp, Homo sapiens
gaaggatgacgtggacatgct

SEQ ID NO: 175 - DNA, 19 bp, Homo sapiens
gatggtgggcatgggtcag

SEQ ID NO: 176 - DNA, 22 bp, Homo sapiens
cttaatgtcacgcacgatttcc

SEQ ID NO: 177 - DNA, 22 bp, Homo sapiens
aaactgtatgacacggccatgg

SEQ ID NO: 178 - DNA, 24 bp, Homo sapiens
ctcctccctgtatttctccgactc

SEQ ID NO: 179 - DNA, 22 bp, Homo sapiens
accatgccattgacatcatctg

SEQ ID NO: 180 - DNA, 22 bp, Homo sapiens
aagacaaccaggtcagcgtcag

SEQ ID NO: 181 - DNA, 31 bp, Homo sapiens; identical to SEQ ID NO: 61

SEQ ID NO: 182 - DNA, 31 bp, Homo sapiens; identical to SEQ ID NO: 60

SEQ ID NO: 183 - DNA, 22 bp, Homo sapiens
cagaaaagaaccggcactgtct

SEQ ID NO: 184 - DNA, 19 bp, Homo sapiens
tgtggtgcagcctctcgat

SEQ ID NO: 185 - DNA, 20 bp, Homo sapiens
ctggacatcgccccgcatgc

SEQ ID NO: 186 - DNA, 24 bp, Homo sapiens
gaggaatcagatgaggatatggga

SEQ ID NO: 187 - DNA, 20 bp, Homo sapiens
aagcaggctgacttggttgc

SEQ ID NO: 188 - DNA, 26 bp, Homo sapiens
tcggtctcttcgactaatcccgccaa

SEQ ID NO: 189 - DNA, 7268 bp, Homo sapiens - Base pairs nucleotide sequence
gaggcgggaggcgccaaggcggagctgctgctcaagctgctcttggccaaggagcgggaccacttccaggacctgcgggcggcgctggagaagacgcagcctcacctgctgcccattctctacctgaacggcgtcgtcgggccgccgcagcccgccgaaggcgcgggttctacctacagcgtcctgtccaccatgccctcagactcagaaagcagcagctccctcagcagtgtgggcactaccgggaaggcgccgtccccaccacccctcctcactgaccagcaagtgaatgagaaggtggagaacctctccattcagctgcggctgatgacccgggagagaaacgagctccgcaagcgcctggcctttgctacgcatggcacggcctttgacaagaggccctaccacaggctgaatcctgactatgagaggctgaagatccagtgcgtgcgagccatgtcggacctgcagagcctgcagaaccagcacaccaacgccttgaagaggtgtgaggaggtggccaaggagactgacttctaccacacactccacagccggctcctgagtgaccagactcggctgaaggatgacgtggacatgctgaggcgggagaatgggcagctgctgcgggagcgaaacctgctgcagcagtcatgggaggacatgaagcggctccacgaggaggaccagaaggagatcggtgacctccgtgcccagcagcagcaggtgttgaagcacaacgggtcatccgagattctcaacaaactgtatgacacggccatggacaagttggaggtggtcaagaaggactatgacgcccttcggaagaggtacagtgagaaagtcgccatccacaatgcagacctgagccgcctggagcagctgggggaggagaaccagcggttgctgaagcagacagagatgctgacccagcagagggacacggccatccagctgcagcaccagtgcgccctctccctgaggaggtttgaggcgatccaccatgagctgaacaaggccacggcgcagaacaaggacctgcagtgggagatggagctgctgcagtcagagctgaccgagctgagaaccacgcaggtgaagacagcaaaggagtcggagaaatacagggaggagcgggacgctgtgtacagcgagtacaagctcatcatgagtgagcgtgaccaggtcatctctgagctggacaagctgcagaccgaagtggagctggccgagtccaagctcaagagcagcacatctgagaagaaggcggccaatgaggagatggaggcgctgcggcagatcaaagacacggtgacaatggatgctgggagagccaacaaggaggttgaaatccttcgaaagcagtgcaaggctctgtgccaggagctgaaggaagccctccaggaggcggatgtggccaagtgccggcgggactgggccttccaggagcgagacaagattgtagcagagcgtgacagcatccggacactgtgtgacaacctgaggcgggagcgggaccgtgcggtgagcgagctggctgaggccctgcgcagcctggatgacacccgcaagcagaagaatgatgtcagccgcgagctgaaggagctcaaggaacagatggaatcccagttggaaaaggaggcccggttccgacagctgatggcccacagctcccacgactcggccattgacacggattccatggagtgggaaacggaagttgtagagttcgagagggagacggaggatattgacttgaaggcactggggtttgatatggcagaaggtgtgaatgagccttgtttcccgggggactgtggcatatttgtcactaaagtggacaaaggaagcattgctgatggccgcttaagggtcaatgactggctgctgagaatcaacgatgtggacctcatcaacaaggacaagaagcaggccatcaaggcgctcctcaatggggagggggccatcaacatggtcgtgcggcggaggaagtccctgggtgggaaggtggtcacgccgctgcacatcaacctcagtggacagaaagacagtggcatcagtctggagaatggagtgtatgctgccgctgtgctgcctggaagccctgccgctaaagaagggtcccttgctgtgggagacaggatcgttgcgatcaatggcattgcactggacaacaagtctctgaatgaatgtgaatctctgctgcggagctgccaggactccctgaccctgtccctcctgaaggtattccctcagagctcctcgtggagtggccagaacatttttgaaaatatcaaagactctgataagatgctgagttttcgagcccatggcccggaggtccaggctcataacaaacggaacttgatacagcacaataactccacgcagacagacatcttctacacggacaggctggaagacaggaaggagccaggccccccaggaggcagcagctcctttctgcataagccattccctgggggacccttgcaggtctgcccccaggcctgtcccagtgcctctgagcgtagcctgagctccttccgctcagatgcctctggggaccgtggctttgggctggtggacgtgcgtggccggcggccactgctgccctttgagaccgaggtgggcccctgtggggttggggaggcctccctggacaaggcagactctgaaggctccaacagcggcgggacctggcccaaggccatgctcagctccacggcagtgcctgagaagctctctgtttataaaaagccaaagcaaagaaagtccatctttgaccctaacactttcaaacgcccccagacaccccccaaaatagactacctgcttccaggtcctgggcctgctcactctccccagccctccaagagggcggggcctctgacacccccaaaacctcccagaaggagcgactccattaagttccagcacaggctggagactagctccgagtcagaagccactctggtgggcagctccccatccactagtcccccgagcgccctgccccctgacgtggaccccggggagcccatgcacgcatcaccccctcgcaaggccagggtccgcattgcttccagctactaccctgaaggagatggggactcctcccacctgccggccaagaaatcctgtgatgaggacctcacctcccagaaggtggatgagctggggcagaagcgtcgccggccaaaatctgctcccagttttcggccgaagcttgctccagtagtgattcctgctcagttcctggaggaacagaagtgtgtcccggccagtggagaactctccccggagctccaggagtgggcaccttactcgcctgggcattccagccggcacagcaaccccccgctataccctagcaggccgtctgtgggcactgttccccggagtttgacccccagcaccactgtgagctccatcctgcggaaccccatctacactgtgcgcagtcacagggtcggcccctgcagctctccacctgcggcccgagatgctggcccccagggtttgcatcccagtgtccagcaccagggacgcctgagcctggacctgagccacaggacctgcagcgactactccgagatgagagccacccatgggtccaactcactgccctccagcgcccgcctgggttcttcgagtaacttgcagttcaaggcggaacgcattaaaatcccatcaacaccaagatatccgcggagtgtcgtgggctccgagagaggttcagtgtcacattctgaatgcagcactcctccacagtcacccctgaacatcgacaccctgtcctcttgtagccagtcccagacctcagcctccacattgcccagaatcgctgtcaaccccgcgtccctcggggagcggagaaaggacaggccttatgtggaggagccacgccacgtgaaggtgcagaagggctcagagccgctgggcatctccatcgtgagtggagagaagggcggcatctacgtctccaaggtgaccgtggggagcatcgctcaccaggctggcctcgagtatggggatcagttactggagttcaacggcataaacctgcggagcgccacggagcagcaggcgcggctcatcatcgggcagcagtgtgataccatcaccatcctggcccagtacaacccccacgtgcaccagctcagcagccactcccggtccagctcacacctggaccctgccggtacccactccactctccagggcagtggcaccaccaccccggagcatccatctgtcatcgacccactgatggagcaggacgaggggcctagcacccccccagccaagcagagcagctccaggattgcgggagatgccaacaagaagaccctggagccacgcgttgtcttcatcaaaaagtcccagctggagcttggggtgcacttgtgtggtgggaacctgcatggggtgtttgtggccgaggtggaggatgacagtcctgccaagggtcctgacggcctcgtgccaggggacctcatcctggagtatggcagcctggacgtgcggaacaagacagtggaggaagtctatgtggagatgctgaagcccagggatggcgtccgcctgaaggtgcagtaccgccctgaggagttcacgaaggccaagggcctgcctggtgacagcttctacatcagggccctgtacgaccggctggcagatgtggagcaagagttgagctttaagaaggacgacatcctctacgtggatgacaccttaccccagggcacgttcgggtcctggatggcttggcagctggacgagaatgcccagaagatccagcgcgggcagattcccagcaaatatgtgatggaccaagaattctccaggaggctcagcatgtctgaagtcaaagatgacaatagcgccacaaagacgctgtcagcggctgcacgccggtccttttttcggaggaaacacaagcacaaacgcagcgggtccaaggacgggaaagacctgctcgccttggatgccttttccagtgactccattccactctttgaagattcggtgagcctggcctatcagcgggtccagaaggtggactgcaccgctctgaggcctgtcctgattctggggcctttgctggacgtggtgaaggagatgctggtgaatgaggctcctggcaagttctgcagatgtccccttgaggtgatgaaggcctcccagcaggccattgagcggggtgtcaaagattgcctgtttgtcgactataagcggagaagcggccatttcgatgtgaccactgtggcgtcaataaaggagatcacagaaaagaaccgacactgcctcctggacattgctccgcacgctattgagcggctccaccacatgcacatctaccccattgtcatcttcatccactacaagagcgccaagcacatcaaggagcagagagaccccatctacctgagggacaaggtgactcagaggcattccaaagagcagtttgaggcggcgcagaagcttgagcaggagtacagcaggtacttcacaggggtcatccagggaggagccctgtcaagcatttgcactcagatcttggcaatggtcaatcaagaacaaaataaagtcctgtggattccagcctgcccgctctaggagaatgctgtgctgtggatgactgcagctggccgcctgaggggacaccagactcagctcttttctagcgactgaaagtagaagtctgtccgtctatgaacatgcgggggaaggatccggaaccaggacccagaagcacctcctttgtagacagagggccacggctgcgtgcgatccaggcccaggcccacacactctgcccgtgtcacacgtgtgctttaacacaaaacagataacactaaagacgggttcagcacccacctttctttagccagctgatcagagatgctgcaaagagaacctttcggatcactcgtttacaagccttttctaagtatttggtggtttatgtttacttgaacggctccatgttgccggtgcccagcccctgtcccctctgtcaaccccctgtcgctttggtgttggtttcgttcccgtcttcagcaaaacgaccttggaacctcaatgggggctgctttgctttgggaggttcttgttggtgggaccagagctttgacaaacctcctgctccttggtggcacctctcctggaaggacgtcacaactccaggtgctcagactgcctgtggcagcagaaccagtgcctttggcattttcctcccacaatggggaaggtgactttggcattcttacaaactcgtctctcggcctttctctcctgccttccacagcctctcgtttctcctccatctgtgcttattacttgaggactgtgtctgctccgtgagagctgcgtgggcagggctgcagtggggtccaggtggtgttcagctgtgctgatgcctgccattgggtcctccttaggctctgtaagtcgtgacagccttcatcagtgcaatgtttgcagggtaattcttaaactttttagagggtggcaggtacatcagttctttttgatatgaaaacattcatgtttcagacattgaattgagagcttttaggggaagcataatggttattgtcactatcaacagtctaaaaagaaaaactgaggtctttttaatcttgattacagcactcacggcatgcaccctactcagtgtgggtgtcttcgtttgggggctttttttttttttgcacttctgaggctagatatgtctggctgaagatttgatgtggttcctccttaagctatgcgtcctgttaataataggtactgtactgggctctgtgtaagtgtcgttggggtaggacctatattttaatactgttcctaacatttcattttactagcgagaaatctttgatttcattttattctttgtaattctagacactagattgtagtttagccataactgatgttttttaaaaagggatatattttcttgcacagttgttcaaaaaagagacaagtttcagtcctcaatgctgtcctttgttttacaggtacaagttttctagctcagacaaactatgaaaaactgtagactattctcaaggtattaactcgcagaccctctgggggtaggggctgttttctaagttacaggcagagtgggactgagatggtacagtgtgcacagacaggtactgagctgacagactgggattttctgtactaaaatgttactttgtataaaagttaaacaggctttagtacaacaaataaaggtcaatttctgt

SEQ ID NO: 190 - protein, 1809 aa, Homo Sapiens - peptide sequence of amino acids
MPSDSESSSSLSSVGTTGKAPSPPPLLTDQQVNEKVENLSIQLRLMTRERNELRKRLAFATHGTAFDKRPYHRLNPDYERLKIQCVRAMSDLQSLQNQHTNALKRCEEVAKETDFYHTLHSRLLSDQTRLKDDVDMLRRENGQLLRERNLLQQSWEDMKRLHEEDQKEIGDLRAQQQQVLKHNGSSEILNKLYDTAMDKLEVVKKDYDALRKRYSEKVAIHNADLSRLEQLGEENQRLLKQTEMLTQQRDTAIQLQHQCALSLRRFEAIHHELNKATAQNKDLQWEMELLQSELTELRTTQVKTAKESEKYREERDAVYSEYKLIMSERDQVISELDKLQTEVELAESKLKSSTSEKKAANEEMEALRQIKDTVTMDAGRANKEVEILRKQCKALCQELKEALQEADVAKCRRDWAFQERDKIVAERDSIRTLCDNLRRERDRAVSELAEALRSLDDTRKQKNDVSRELKELKEQMESQLEKEARFRQLMAHSSHDSAIDTDSMEWETEVVEFERETEDIDLKALGFDMAEGVNEPCFPGDCGIFVTKVDKGSIADGRLRVNDWLLRINDVDLINKDKKQAIKALLNGEGAINMVVRRRKSLGGKVVTPLHINLSGQKDSGISLENGVYAAAVLPGSPAAKEGSLAVGDRIVAINGIALDNKSLNECESLLRSCQDSLTLSLLKVFPQSSSWSGQNIFENIKDSDKMLSFRAHGPEVQAHNKRNLIQHNNSTQTDIFYTDRLEDRKEPGPPGGSSSFLHKPFPGGPLQVCPQACPSASERSLSSFRSDASGDRGFGLVDVRGRRPLLPFETEVGPCGVGEASLDKADSEGSNSGGTWPKAMLSSTAVPEKLSVYKKPKQRKSIFDPNTFKRPQTPPKIDYLLPGPGPAHSPQPSKRAGPLTPPKPPRRSDSIKFQHRLETSSESEATLVGSSPSTSPPSALPPDVDPGEPMHASPPRKARVRIASSYYPEGDGDSSHLPAKKSCDEDLTSQKVDELGQKRRRPKSAPSFRPKLAPVVIPAQFLEEQKCVPASGELSPELQEWAPYSPGHSSRHSNPPLYPSRPSVGTVPRSLTPSTTVSSILRNPIYTVRSHRVGPCSSPPAARDAGPQGLHPSVQHQGRLSLDLSHRTCSDYSEMRATHGSNSLPSSARLGSSSNLQFKAERIKIPSTPRYPRSVVGSERGSVSHSECSTPPQSPLNIDTLSSCSQSQTSASTLPRIAVNPASLGERRKDRPYVEEPRHVKVQKGSEPLGISIVSGEKGGIYVSKVTVGSIAHQAGLEYGDQLLEFNGINLRSATEQQARLIIGQQCDTITILAQYNPHVHQLSSHSRSSSHLDPAGTHSTLQGSGTTTPEHPSVIDPLMEQDEGPSTPPAKQSSSRIAGDANKKTLEPRVVFIKKSQLELGVHLCGGNLHGVFVAEVEDDSPAKGPDGLVPGDLILEYGSLDVRNKTVEEVYVEMLKPRDGVRLKVQYRPEEFTKAKGLPGDSFYIRALYDRLADVEQELSFKKDDILYVDDTLPQGTFGSWMAWQLDENAQKIQRGQIPSKYVMDQEFSRRLSMSEVKDDNSATKTLSAAARRSFFRRKHKHKRSGSKDGKDLLALDAFSSDSIPLFEDSVSLAYQRVQKVDCTALRPVLILGPLLDVVKEMLVNEAPGKFCRCPLEVMKASQQAIERGVKDCLFVDYKRRSGHFDVTTVASIKEITEKNRHCLLDIAPHAIERLHHMHIYPIVIFIHYKSAKHIKEQRDPIYLRDKVTQRHSKEQFEAAQKLEQEYSRYFTGVIQGGALSSICTQILAMVNQEQNKVLWIPACPL

SEQ ID NO: 191 - DNA, 21 bp, Homo sapiens
gactgatgatcagctggcttg

(19) United States
(12) Patent Application Publication
(10) Pub. No.: US 2006/0100132 A1
(43) Pub. Date: May 11, 2006

(54) Method for diagnosing inflammatory bowel disease

(76) Inventors: Brit Corneliussen, Molndal (SE); Stefan Schreiber, Kiel (DE); Monika Stoll, Munster (DE)

Correspondence Address: WHITE & CASE LLP;PATENT DEPARTMENT, 1155 AVENUE OF THE AMERICAS, NEW YORK, NY, 10036, US

(21) Appl. No.: 10/559,415

(22) PCT Filed: Jun. 3, 2004
(86) PCT No.: PCT/SE04/00861

§ 371 Date: Dec. 6, 2005

(30) Foreign Application Priority Data
Jun. 6, 2003 (GB) 0313081.2
Nov. 26, 2003 (GB) 0327427.1

(51) Int. Cl. C12Q 1/68; G01N 33/53; A61K 38/17

(52) U.S. Cl. 514/2; 435/6; 435/7.1

(57) ABSTRACT

The present invention relates to a method for identifying a compound capable of modulating the action of the DLG5 protein which method comprises subjecting one or more test compounds to a screen comprising a polypeptide containing the amino acid sequence shown in SEQ ID NO: 2, or a homologue thereof or a fragment of either. Such compounds may be useful in the treatment of inflammatory bowel disease. The present invention also relates to a method for diagnosing inflammatory bowel disease by detecting variant DLG5.

### TECHNICAL FIELD

The inventors have discovered a human gene linked to susceptibility to inflammatory bowel disease (IBD) using linkage and association analysis. The present invention therefore relates to diagnostic techniques for the detection of IBD, and for determining a patient's susceptibility to develop IBD by detecting all or part of this gene, its precursors or products (e.g. mRNA, cDNA, genomic DNA, or protein). The invention is also directed to methods for identifying modulators of IBD, which modulators, such as chemical compounds, antisense molecules and antibodies modulate the gene identified.

### BACKGROUND TO INVENTION

Inflammatory bowel disease (IBD) is characterised by a chronic relapsing intestinal inflammation of the gastrointestinal tract. It affects ˜ 1/1,000 individuals in Western countries with the median age of onset in early adulthood. To date, the etiology of this disease is unknown. Based on clinical and histopathological features, IBD is categorised into two main subtypes, Crohn's disease (CD) (On Line Mendelian Inheritance in Man—a database produced by Johns Hopkins University available at NCBI, OMIM 266600) and ulcerative colitis (UC) (OMIM 191390). Although the cause of IBD is unknown, both familial clustering of the disease and increased concordance in monozygotic twins shows a strong genetic susceptibility. Estimates of sibling risk (λs) show a range of 10-50, suggesting that genetic factors play a significant role in the predisposition to IBD. In the present context the term IBD is intended to include IBD, as well as Crohn's disease and ulcerative colitis.

Previous genome wide linkage analyses have identified a number of susceptibility locus for IBD, e.g. IBD1 (OMIM 266600) (Hugot et al., Nature. 379:821-823, 1996; Brant et al., Gastroenterlogy 115:1056-1061, 1998; Curran et al., Gastroenterology 115: 1066-1071, 1998; and, Hampe et al., Am. J. Hum. Genet. 64:808-816, 1999a), IBD 2 (OMIM 601458) (Duerr et al., Am. J. Hum. Genet 63:95-100, 1988; and, Parkes et al., Am. J. Hum. Genet. 67:1605-1610, 2000), IBD3 (OMIM 604519) (Hampe et al., Am. J. Hum. Genet. 65:1647-1655, 1999b), IBD7 (OMIM 605225) (Cho et al., Proc. Nat. Acad. Sci. 95:7502-7507; and, Cho et al., Hum. Molec. Genet. 9:1425-1432, 2000).

There is therefore a desire to identify genes with a significant association to the development of IBD. This may enable the development of novel therapies for IBD by screening for compounds and other entities, such as antibodies, which modulate the activity of the proteins encoded by the associated genes. Knowledge of the sequence of the associated genes may also enable the development of novel antigene methods to modulate the expression of the associated gene and may also enable the development of novel gene therapy techniques to treat IBD. The discovery of associated genes may also assist in developing novel methods for diagnosing IBD via (i) analysis of the pattern of genotypes of associated single nucleotide polymorphisms (SNPs), (ii) measuring the levels of the transcribed mRNA present in affected tissue or (iii) measuring the levels of the protein in affected tissue. It is possible that the diagnosis of IBD, or the prediction of predisposition to IBD, by these methods may be achieved in patients who do not yet display the classical symptoms of the disease. Such determination of susceptibility to IBD or the early detection of disease development may lead to earlier clinical intervention than is currently possible and may lead to more effective treatment of the disease.

The present invention is based on our discovery of an association with IBD for a single gene termed dlg5 located on chromosome 10q22.3.

As used herein, the gene is referred to as the dlg1 gene. Specifically, the cDNA sequence is shown in SEQ ID No: 1. Encoded protein is shown in SEQ ID No: 2 and is referred to as DLG5. A C-terminally truncated cDNA sequence is shown in SEQ ID NO: 189 and its encoded protein is shown in SEQ ID NO: 190.

DLG5 belongs to the so-called MAGUK family of proteins (Membrane Associated Guanylate Kinases, reviewed in Dimitratos et al. BioEssays 21:912-921, 1999). Proteins of this family contain several distinct protein motifs including a guanylate kinase domain, one or several PDZ domains (Postsynaptic density 95, Discs large, Zona occludens-1 domain) and a SH3 domain (src homology domain 3). PDZ domains and SH3 domains have been shown to mediate protein-protein interactions. In several cases where PDZ domain interactions have been characterised they have been shown to interact with short C-terminal sequences of membrane proteins (Kreienkamp, Curr. Opin. Pharm. 2:581-586, 2002). SH3 domains have been found to interact with proline rich surface regions of target proteins. Since the guanylate kinase domain for some MAGUK proteins has been shown to mediate protein-protein interactions while it lacks kinase activity, it is generally believed that the main function also for this domain is to mediate protein-protein interactions. Therefore, MAGUK proteins are considered as scaffold proteins, orchestrating signalling molecules to specific membrane locations. Besides establishment of cell polarity of epithelia, MAGUK proteins have also been implicated in establishment of postsynaptic compartments in neurons (Kreienkamp, Curr. Opin. Pharm. 2:581-586, 2002). For example, an interaction between a PDZ domain of the MAGUK protein hDLG1/PSD-95 and the intracellular tail of the NMDA receptor has been identified. Since it has been identified that the C-termini of some 50 intracellular and membrane proteins have high affinity for the PDZ domains of hDLG-1/PSD-95, it has been hypothesised that such clustering of scaffold proteins to multiple membrane receptors is responsible for localisation of neuroreceptors at postsynaptic sites (Kreienkamp, Curr. Opin. Pharm, 2:581-586, 2002).

Until recently, little has been known about the function of DLG5. Partial EST sequences for dlg5 were identified from a database search by its similarity to other MAGUK proteins (Nakamura et al., FEBS 433:63-67, 1998). The authors identified a partial cDNA sequence referred to as P-dlg and showed by immunostaining that the protein was expressed in epithelial gland cells of the prostate. It was also shown by a two-hybrid screen that the DLG5/P-dlg specifically interacted with p55, another MAGUK protein. Northern blot analyses showed variable expression in multiple tissues (Nakamura et al., FEBS 433:63-67, 1998 and Shah et al., BMC Genomics 3:6 2002). Shah et al. also showed that the human gene consisting of 32 exons, encoded a full length DLG5 protein of 1809 amino acids. The DLG5 protein contains 4 PDZ domains followed by an SH3 domain and a C terminal guanylate kinase domain.

It was recently shown that DLG5 could be identified in a two-hybrid screen using vinexin as bait (Wakabayashi et al., JBC, 25th Mar. 20032003). Furthermore, the authors showed that vinexin, DLG5 and β-catenin could form a ternary complex, providing a direct link to the adhesion junction complex in epithelial cells.

In vertebrate gut epithelial cells three types of cell junctions are formed (reviewed in Tsukita et al,. Nature Rev. Mol. Cell. Biol. 2:285-293, 2001). Tight junctions are located towards the apical border of the basolateral side and are considered to function both as a barrier for the extracellular environment as well as a fence for membrane diffusion. Adherence junctions are formed immediately basolateral of tight junctions and their role is less clear than for tight junctions. They are considered to be important for the mechanical strength of cell contacts, but it is also clear that their regulation has to be precisely coordinated with tight junctions, for example when immune cells passes through the epithelial barrier. The mutual dependence between tight junctions and adherence junctions are underscored by the findings that while formation of tight junctions does not occur until adherence junctions are intact, adherence junctions can not form when formation of tight junctions are inhibited by overexpression of a dominant negative mutant of the tight junction MAGUK protein ZO-3 (Wittchen et al., J. Cell. Biol. 151:825-836, 2000; and refs therein). Finally, multiple desmosomes are located along the basolateral sides and are mainly considered to contribute to the mechanical strength of cell contacts.

Many proteins have been shown to be localised to cellular junctions. Membrane proteins, such as for example occludin and claudins, are found at tight junctions while members of the cadherin family mediates cell-cell contacts at adherence junctions. A large number of proteins connect to the cytoplasmatic side of these membrane proteins, linking the complexes both to the actin cytoskeleton and to intracellular signaling. At adherence junctions, the cytoplasmatic part of cadherin binds β-catenin, thus providing a link between DLG5 and adherence junctions.

The background data above strongly supports a functional role for DLG5 in gut epithelial cell function and integrity. The inventors propose that protein(s) encoded by the dlg5 gene, which has only now been identified as being genetically linked to susceptibility to IBD, are directly or indirectly involved in the pathogenesis of gut inflammation.

The inventors have identified 20 unique nucleotide variations within the dlg5 gene, four of these result in codon changes, a further two are deletions.

### SUMMARY OF THE INVENTION

The inventors have identified a gene located on chromosome 10q22.3, termed dlg5, which demonstrates genetic association linkage to susceptibility to IBD. The gene, mRNA (or cDNA prepared therefrom) and protein sequences corresponding to such transcript are therefore diagnostic or prognostic markers of IBD, and can be used to design specific probes, or to generate antibodies, capable of detecting the presence of nucleotide sequence polymorphims or mutations of the gene or mRNA, or of measuring the levels of the mRNA or encoded protein present in a test sample, such as a body fluid or cell sample. In addition the gene and protein encoded thereby is a potential target for therapeutic intervention in IBD disease, for instance in the development of antisense nucleic acid targeted to the mRNA, or transgenic therapies; or more widely in the identification or development of chemical or hormonal therapeutic agents. The person skilled in the art is also capable of devising screening assays to identify compounds (chemical or biological) that modulate (activate or inhibit) the identified gene or encoded protein, which compounds may prove useful as therapeutic agents in treating or preventing IBD.

### DETAILED DESCRIPTION OF THE INVENTION

According to a first aspect of the invention there is provided a method for identifying a compound capable of modulating the action of the DLG5 protein which method comprises subjecting one or more test compounds to a screen comprising a polypeptide containing the amino acid sequence shown in SEQ ID NO: 2, or a homologue thereof or a fragment of either.

The term “fragment” as used herein refers to a subsequence of the full length sequence that comprises at least 25, preferably at least 50, more preferably at least 100 consecutive amino acids of the sequence depicted in SEQ ID NO: 2, preferably the fragment is a polypeptide that is the DLG5 protein with either or both C-terminal and N-terminal truncations, such as the polypeptide depicted in SEQ ID NO: 190.

It is understood that the polypeptide for use in the invention may be both a fragment and a homologue of the DLG5 protein.

In a preferred embodiment, the screening methods of the invention are carried out using a polypeptide comprising an amino acid sequence as depicted in SEQ ID NO: 2, or a sequence possessing, in increasing order of preference, at least 80%, 85%, 90%, 95%, 97%, 98% and 99% amino acid sequence identity thereto. Such variants are herein referred to as “homologues”.

The sequence identity between two sequences can be determined by pair-wise computer alignment analysis, using programs such as, BestFit, Gap or FrameAlign. The preferred alignment tool is BestFit. In practise, when searching for similar/identical sequences to the query search, from within a sequence database, it is generally necessary to perform an initial identification of similar sequences using suitable software such as Blast, Blast2, NCBI Blast2, WashU Blast2, FastA, Fasta3 and PILEUP, and a scoring matrix such as Blosum 62. Such software packages endeavour to closely approximate the “gold-standard” alignment algorithm of Smith-Waterman. Thus, the selected software/search engine programme for use in assessing identity/similarity, i.e how two primary polypeptide sequences line up is Smith-Waterman. Identity refers to direct matches, similarity allows for conservative substitutions.

Allelic variants or versions of the DLG5 protein may exist within the human population, particularly between distinct ethnic groups. A further aspect of the invention involves the selection and use of the appropriate version of the DLG5 protein to be included in screens so as to discover compounds capable of altering the activity of said DLG5 version in vivo. The inventors have identified four codon changing nucleotide polymorphisms within one or other exons of dlg5 gene, each of these, alone or in combination, would provide numerous allelic variant protein versions of DLG5 for use in any aspect of the present invention.

Investigators may wish to screen their compounds against the most prevalent version of the DLG5 protein and also against the less frequent versions of the DLG5 protein in order to detect any differential pharmacological activity between the various versions of the target. A further aspect of the invention is the screening of various ethnic based populations to measure the allele frequencies of the nucleotide polymorphisms in the dlg5 gene within said populations. This information may be of value in estimating the efficacy of new compounds capable of altering the activity of DLG5 within these populations and in particular in estimating the proportion of the population which may not respond to the therapy.

Polymorphism refers to the occurrence of two or more genetically determined alternative alleles or sequences within a population. A polymorphic marker is the site at which divergence occurs. Preferably markers have at least two alleles, each occurring at frequency of greater than 1%, and more preferably at least 10%, 15%, 20%, 30% or more of a selected population.

Single nucleotide polymorphisms (SNP) are generally, as the name implies, single nucleotide or point variations that exist in the nucleic acid sequence of some members of a species. Such polymorphism variation within the species, is generally regarded to be the result of spontaneous mutation throughout evolution. The mutated and normal sequences co-exist within the species' population sometimes in a stable or quasi-stable equilibrium. At other times the mutation may confer some advantage to the species and with time may be incorporated into the genomes of all or a majority of members of the species.

Some SNPs occur in the protein coding sequences, in which case, one of the polymorphic protein forms may possess a different amino acid which may give rise to the expression of a variant protein and, potentially, a genetic disease. These changes in function may be mediated by several mechanisms including, but not limited to, alterations in protein folding, alterations in ligand and substrate binding affinity and alterations in membrane binding affinity and may lead to gain of activity or loss of activity for the protein in vivo. Such alterations in the activity of the protein in vivo may be of clinical significance in the development of IBD. Alteration to the amino acid sequence of the protein may also affect the efficacy of drug therapy for IBD by altering the specificity between protein and compounds selected by screening to modulate its activity. Thus compounds selected by screening may have different efficacies in modulating the activity of protein in different individuals according to the versions of the gene that they carry. In particular an individual who is homozygous for a less common variant of the gene may not respond well to a therapy developed by screening compounds against the dominant variant.

The screening methods according to the invention may be carried out using conventional procedures, for example by bringing the test compound or compounds to be screened and an appropriate substrate into contact with the polypeptide, or a cell capable of producing it, or a cell membrane preparation thereof, and determining affinity for the polypeptide in accordance with standard techniques.

Any compound identified in this way may prove useful in the treatment of IBD in humans and/or other animals. The invention thus extends to a compound selected through its ability to regulate the activity of the DLG5 protein in vivo as primarily determined in a screening assay utilising the polypeptide containing an amino acid sequence shown in SEQ ID NO: 2, or a homologue or fragment thereof, or a gene coding therefore (such as that disclosed in SEQ ID NO: 1) for use in the treatment of a disease in which the over- or under-activity or unregulated activity of the protein is implicated.

According to a further aspect of the invention there is provided a screening assay or method for identifying potential anti-IBD therapeutic compounds comprising contacting an assay system capable of detecting the effect of a test compound against expression level of DLG5, with a test compound and assessing the change in expression level of DLG5.

Compounds that modulate the expression of DNA or RNA of DLG5 polypeptides may be detected by a variety of assay systems. A suitable assay system may be a simple “yes/no” assay to determine whether there is a change in expression of a reporter gene, such as beta-galactosidase, luciferase, green fluorescent protein or others known to the person skilled in the art (reviewed by Naylor, Biochem. Pharmacol. 58:749-57, 1999). The assay system may be made quantitative by comparing the expression or function of a test sample with the levels of expression or function in a standard sample. Systems in which transcription factors are used to stimulate a positive output, such as transcription of a reporter gene, are generally referred to as “one-hybrid systems” (Wang, M. M. and Reed, R. R. (1993) Nature 364:121-126). Using a transcription factor to stimulate a negative output (growth inhibition) may thus be referred to as a “reverse one-hybrid system” (Vidal et al, 1996, supra). Therefore, in an embodiment of the present invention, a reporter gene is placed under the control of the dlg5 promoter. A suitable dlg5 promoter sequence is disclosed in SEQ ID NO: 5.

In a further aspect of the invention we provide a cell or cell line comprising a reporter gene under the control of the dlg5 promoter.

According to another aspect of the present invention there is provided a method of screening for a compound potentially useful for treatment of IBD, which comprises assaying the compound for its ability to modulate the activity or amount of DLG5. Preferably the assay is selected from:
(i) measurement of DLG5 activity using a cell line which expresses the DLG5 polypeptide or using purified DLG5 polypeptide; and
(ii) measurement of dlg5 transcription or translation in a cell line expressing the DLG5 polypeptide.

The “DLG5 polypeptide” refers to the DLG5 protein, a homologue thereof, or a fragment of either.

Thus, in a further aspect of the invention, cell cultures expressing the DLG5 polypeptide can be used in a screen for therapeutic agents. Effects of test compounds may be assayed by changes in mRNA or protein of DLG5. As described below, cells (i.e. mammalian, bacterial, yeast etc.) can be engineered to express the DLG5 polypeptide.

Thus, according to a further aspect of the invention there is provided a method of testing potential therapeutic agents for the ability to suppress IBD phenotype comprising contacting a test compound with a cell engineered to express the DLG5 polypeptide; and determining whether said test compound modulates expression of the DLG5 polypeptide.

We also provide a method for identifying inhibitors of transcription of dlg5, which method comprises contacting a potential therapeutic agent with a cell or cell line as described above and determining inhibition of dlg5 transcription by the potential therapeutic agent by reference to a lack of or reduction in expression of the reporter gene.

Any convenient test compound or library of test compounds may be used in conjunction with the test assay. Particular test compounds include low molecular weight chemical compounds (preferably with a molecular weight less than 1500 daltons) suitable as pharmaceutical or veterinary agents for human or animal use, or compounds for non-administered use such as cleaning/sterilising agents or for agricultural use. Test compounds may also be biological in nature, such as antibodies.

According to a further aspect of the invention there is provided a compound identified by a screening method as defined herein.

According to another aspect of the present invention there is provided use of a compound able to modulate the activity or amount of DLG5 in the preparation of a medicament for the treatment of IBD. Modulation of the amount of DLG5 by a compound may be brought about for example through altered gene expression level or message stability. Modulation of the activity of DLG5 by a compound may also be brought about for example through compound binding to the DLG5 protein. In one embodiment, modulation of DLG5 comprises use of a compound able to reduce the activity or amount of DLG5. In another embodiment, modulation of DLG5 comprises use of a compound able to increase the activity or amount of DLG5.

It will be appreciated that the term ‘for the treatment of IBD’, and variations thereon, includes therapeutic and prophylactic (preventative) treatment.

According to another aspect of the present invention there is provided a method of preparing a pharmaceutical composition which comprises:
i) identifying a compound as useful for treatment of IBD according to a screening method as described herein; and
ii) mixing the compound or a pharmaceutically acceptable salt thereof with a pharmaceutically acceptable excipient or diluent.

According to a further aspect of the invention there is provided a method of treatment of a patient suffering from IBD comprising administration to said patient of an effective amount of a compound identified according to a screening method of the invention or a composition prepared by the method described herein.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient, which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial BIBDrd), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial BIBDrd), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

Having identified that the dlg5 gene is implicated in IBD, this presents many molecular diagnostic opportunities. It is known to persons skilled in the art that clinically significant information may be obtained by the measurement of the levels of nucleic acids, proteins or other analytes that occur within biological samples. When nucleic acids, proteins or other analytes occur in polymorphic form then there may also be diagnostic utility in by identifying which of the various versions of said polymorphic nucleic acids, proteins or other analytes occur within a sample.

An investigator may wish to measure the levels of DLG5 protein or to measure the levels of dlg5 mRNA transcript present in a sample. An investigator may also wish to perform nucleic acid sequence analyses to detect variant nucleotides present within the sample, these analyses may be performed on either the DNA or RNA fraction of the sample and are well known to the person skilled in the art. An investigator may also wish to perform protein sequence analysis either directly, by degradation based techniques which are well known in the art, or indirectly by molecular recognition techniques including immunoassay, or by techniques based on detecting changes in the physical characteristics of the protein such as functional or substrate specificity assays or iso-electric focusing.

According to a further aspect of the invention there is provided a method for diagnosing or prognosing or monitoring IBD, comprising testing a biological sample for aberrant levels of DLG5.

The term “aberrant levels” refers to levels that are outside the normal range. The normal range can be determined by testing many normal tissues or may be determined from a side by side comparison of the test sample with the normal or control sample. For the purposes of this application, aberrant expression refers to a 1.5-fold difference or more in level of nucleic acid in a disease sample compared to control normal. Nucleic acid as used herein refers to both RNA and DNA.

The test biological sample is conveniently a sample of sinovial fluid, blood, buccal scrape, urine or other body fluid or tissue obtained from an individual.

The invention lies in the identification of the gene identified herein being linked to IBD disease prevalence. Accordingly, in part, the invention is directed to any diagnostic method capable of assessing the differential expression level, relative to expression in control tissues, of the dlg5 gene identified herein, either alone or as a panel. In particular, such methods include assessment of mRNA transcript levels and/or protein levels. The presence of aberrant expression levels of the gene indicating the presence of IBD or an increased likelihood to develop the disorder.

As noted above, in one embodiment the diagnostic/detection methods of the invention are employed to detect the presence of one or more SNPs or small insertions, deletions or duplications of DLG5 or dlg5. Suitable SNPs and deletions of DLG5 or dlg5 include those identified in Table 3.

Knowledge of polymorphisms can be of assistance in identifying patients susceptible to particular diseases and those most suited to therapy with particular pharmaceutical agents (the latter is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43:254; Marshall (1997), Nature Biotechnology. 15:1249; International Patent Application WO 97/40462, Spectra Biomedical; and Schafer et al, (1998), Nature Biotechnology. 16:33.

A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that nucleotide sequence polymorphisms with low frequency alleles are particularly useful as markers of low frequency haplotypes. As particular mutations or polymorphisms associated with certain clinical features, such as adverse or abnormal events, are likely to be of low frequency within the population, low frequency nucleotide sequence variations may be particularly useful in identifying these mutations (for examples see: Linkage disequilibrium at the cystathionine beta synthase (CBS) locus and the association between genetic variation at the CBS locus and plasma levels of homocysteine (De Stefano et al., Ann Hum Genet (1998) 62:481-90; and, Keightley et al., Blood (1999) 93:4277-83).

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K).

The presence of a particular nucleic acid base at a polymorphism position will be represented by the base following the polymorphism position. For (a hypothetical) example, the presence of adenine at position 300 will be represented as: 300A.

We provide examples of nucleotide polymorphisms, including those that affect the amino acid sequence of the DLG5 protein. Such amino acid changing polymorphisms are indicated in Table 3 as “non-synonymous”.

Nucleotide polymorphisms (mutations) in the promoter and UTR regions may also affect the transcription and expression of the dlg5 gene leading to either increased or decreased levels of expression or to unregulated activity of the DLG5 protein in vivo. Such alterations in the level of expression of the DLG5 protein in vivo may result in a gain or loss of function, which is of clinical significance. Recently, it has been reported that even polymorphisms that do not result in an amino acid change can cause different structural folds of mRNA with potentially different biological functions (Shen et al., (1999) Proc Natl Acad Sci USA 96:7871-7876).

In one embodiment of the invention the screening methods described herein utilise a DLG5 protein variant which is transcribed from a nucleic acid sequence based on that shown in SEQ ID NO:1 or 6.

Nucleotide polymorphisms within dlg5 or DLG5 may also be used as diagnostic markers of predisposition to disease. Genotyping nucleotide sequence variants in populations suffering from IBD and in control populations not suffering from IBD but matched for factors including, but not limited to, racial ancestry, country of origin, sex, age and body mass index may allow investigators to identify increased risk factors associated with the development of IBD disease according to the inheritance of certain SNP genotypes or haplotypes which are more prevalent in populations with IBD compared to their incidence in the corresponding control populations. This may enable screening for individuals at increased risk of developing IBD by measuring the genotypes and haplotypes of these nucleotide sequence polymorphisms within non-symptomatic individuals. We have discovered novel sequence polymorphisms in the dlg5 gene which may be useful for the diagnosis of IBD. Public domain nucleotide sequence variations, which may also be useful for the diagnosis of IBD, or for research into IBD, are also identified herein. Table 3 lists the sequence polymorphisms in dlg5. Those database-derived polymorphisms present in the public domain are annotated as either rs- or tsc-. The other SNPs, annotated DLGe, are believed to be identified herein, for the first time. Tsc-stands for the SNP consortium.

A nucleotide sequence variation or polymorhisms could be a single nucleotide polymorhism, a deletion of one or several nucleotides, a duplication of one or several nucleotides or an insertion of one or several nucleotides in the nucleotide sequence of the gene or in sequences modulating the expression of the dlg5 gene.

According to one aspect of the present invention there is provided a method for the diagnosis of a single nucleotide polymorphism associated with IBD, which method comprises determining from human nucleic acid, the identity of the nucleotide at position 16 according to one or more of SEQ ID NOs: 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91 and 93; and determining the status of the human by reference to polymorphism(s) detected. With respect to the mutation disclosed in SEQ ID NO: 32 and 33, the nucleotide at position 16 will either be C, or in the allele with the 7-base deletion, a G.

The term human includes both a human having or suspected of having inflammatory bowel disease and an asymptomatic human who may be tested for predisposition or susceptibility to IBD. At each position the human may be homozygous for an allele or the human may be a heterozygote.

In one embodiment of the invention preferably the method for diagnosis described herein is one in which the single nucleotide polymorphism located at position 16 (according to SEQ ID NO: 7) is the presence of G and/or A.

In one embodiment of the invention preferably the method for diagnosis described herein is one in which the single nucleotide polymorphism located at position 16 (according to SEQ ID NO: 9) is the presence of C and/or T.

In one embodiment of the invention preferably the method for diagnosis described herein is one in which the single nucleotide polymorphism located at position 16 (according to SEQ ID NO: 11) is the presence of C and/or T.

In one embodiment of the invention preferably the method for diagnosis described herein is one in which the single nucleotide polymorphism located at position 16 (according to SEQ ID NO: 13) is the presence of G and/or A.

In one embodiment of the invention preferably the method for diagnosis described herein is one in which the single nucleotide polymorphism located at position 16 (according to SEQ ID NO: 15) is the presence of G and/or C.

In one embodiment of the invention preferably the method for diagnosis described herein is one in which the single nucleotide polymorphism located at position 16 (according to SEQ ID NO: 17) is the presence of C and/or T.

In one embodiment of the invention preferably the method for diagnosis described herein is one in which the single nucleotide polymorphism located at position 16 (according to SEQ ID NO: 19) is the presence of A and/or G.

In one embodiment of the invention preferably the method for diagnosis described herein is one in which the single nucleotide polymorphism located at position 16 (according to SEQ ID NO: 21) is the presence of C and/or A.

In one embodiment of the invention preferably the method for diagnosis described herein is one in which the single nucleotide polymorphism located at position 16 (according to SEQ ID NO: 23) is the presence of C and/or A.

In one embodiment of the invention preferably the method for diagnosis described herein is one in which the single nucleotide polymorphism located at position 16 (according to SEQ ID NO: 25) is the presence of G and/or A.

In one embodiment of the invention preferably the method for diagnosis described herein is one in which the single nucleotide polymorphism located at position 16 (according to SEQ ID NO: 27) is the presence of C and/or T.

In one embodiment of the invention preferably the method for diagnosis described herein is one in which the single nucleotide polymorphism located at position 16 (according to SEQ ID NO: 29) is the presence of C and/or G.

In one embodiment of the invention preferably the method for diagnosis described herein is one in which the single nucleotide polymorphism located at position 16 (according to SEQ ID NO: 3 1) is the presence of C and/or T.

In one embodiment of the invention preferably the method for diagnosis described herein is one in which the single nucleotide polymorphism located at position 16 (according to SEQ ID NO: 35) is the presence of G and/or A.

In one embodiment of the invention preferably the method for diagnosis described herein is one in which the single nucleotide polymorphism located at position 16 (according to SEQ ID NO: 37) is the presence of G and/or C.

In one embodiment of the invention preferably the method for diagnosis described herein is one in which the single nucleotide polymorphism located at position 16 (according to SEQ ID NO: 39) is the presence of G and/or A.

In one embodiment of the invention preferably the method for diagnosis described herein is one in which the single nucleotide polymorphism located at position 16 (according to SEQ ID NO: 41) is the presence of C and/or T.

In one embodiment of the invention preferably the method for diagnosis described herein is one in which the single nucleotide polymorphism located at position 16 (according to SEQ ID NO: 43) is the presence of G and/or A.

In one embodiment of the invention preferably the method for diagnosis described herein is one in which the single nucleotide polymorphism located at position 16 (according to SEQ ID NO: 45) is the presence of C and/or T.

In one embodiment of the invention preferably the method for diagnosis described herein is one in which the single nucleotide polymorphism located at position 16 (according to SEQ ID NO: 47) is the presence of C and/or T.

In one embodiment of the invention preferably the method for diagnosis described herein is one in which the single nucleotide polymorphism located at position 16 (according to SEQ ID NO: 49) is the presence of C and/or T.

In one embodiment of the invention preferably the method for diagnosis described herein is one in which the single nucleotide polymorphism located at position 16 (according to SEQ ID NO: 51) is the presence of C and/or T.

In one embodiment of the invention preferably the method for diagnosis described herein is one in which the single nucleotide polymorphism located at position 16 (according to SEQ ID NO: 53) is the presence of G and/or C.

In one embodiment of the invention preferably the method for diagnosis described herein is one in which the single nucleotide polymorphism located at position 16 (according to SEQ ID NO: 55) is the presence of C and/or T.

In one embodiment of the invention preferably the method for diagnosis described herein is one in which the single nucleotide polymorphism located at position 16 (according to SEQ ID NO: 57) is the presence of G and/or C.

In one embodiment of the invention preferably the method for diagnosis described herein is one in which the single nucleotide polymorphism located at position 16 (according to SEQ ID NO: 59) is the presence of C and/or T.

In one embodiment of the invention preferably the method for diagnosis described herein is one in which the single nucleotide polymorphism located at position 16 (according to SEQ ID NO: 61) is the presence of C and/or A.

In one embodiment of the invention preferably the method for diagnosis described herein is one in which the single nucleotide polymorphism located at position 16 (according to SEQ ED NO: 63) is the presence of T and/or A.

In one embodiment of the invention preferably the method for diagnosis described herein is one in which the single nucleotide polymorphism located at position 16 (according to SEQ ID NO: 65) is the presence of C and/or G.

In one embodiment of the invention preferably the method for diagnosis described herein is one in which the single nucleotide polymorphism located at position 16 (according to SEQ ID NO: 67) is the presence of G and/or A.

In one embodiment of the invention preferably the method for diagnosis described herein is one in which the single nucleotide polymorphism located at position 16 (according to SEQ ID NO: 69) is the presence of C and/or T.

In one embodiment of the invention preferably the method for diagnosis described herein is one in which the single nucleotide polymorphism located at position 16 (according to SEQ ID NO: 71) is the presence of A and/or (3 (as a result of a single base deletion).

In one embodiment of the invention preferably the method for diagnosis described herein is one in which the single nucleotide polymorphism located at position 16 (according to SEQ ID NO: 73) is the presence of C and/or T.

In one embodiment of the invention preferably the method for diagnosis described herein is one in which the single nucleotide polymorphism located at position 16 (according to SEQ ID NO: 75) is the presence of C and/or T.

In one embodiment of the invention preferably the method for diagnosis described herein is one in which the single nucleotide polymorphism located at position 16 (according to SEQ ID NO: 77) is the presence of C and/or A.

In one embodiment of the invention preferably the method for diagnosis described herein is one in which the single nucleotide polymorphism located at position 16 (according to SEQ ID NO: 79) is the presence of C and/or T.

In one embodiment of the invention preferably the method for diagnosis described herein is one in which the single nucleotide polymorphism located at position 16 (according to SEQ ID NO: 81) is the presence of C and/or T.

In one embodiment of the invention preferably the method for diagnosis described herein is one in which the single nucleotide polymorphism located at position 16 (according to SEQ ID NO: 83) is the presence of G and/or A.

In one embodiment of the invention preferably the method for diagnosis described herein is one in which the single nucleotide polymorphism located at position 16 (according to SEQ ID NO: 85) is the presence of C and/or T.

In one embodiment of the invention preferably the method for diagnosis described herein is one in which the single nucleotide polymorphism located at position 16 (according to SEQ ID NO: 87) is the presence of A and/or G.

In one embodiment of the invention preferably the method for diagnosis described herein is one in which the single nucleotide polymorphism located at position 16 (according to SEQ ID NO: 89) is the presence of G and/or A.

In one embodiment of the invention preferably the method for diagnosis described herein is one in which the single nucleotide polymorphism located at position 16 (according to SEQ ID NO: 91) is the presence of C and/or G.

In one embodiment of the invention preferably the method for diagnosis described herein is one in which the single nucleotide polymorphism located at position 16 (according to SEQ ID NO: 93) is the presence of G and/or A.

In one embodiment of the invention preferably the method for diagnosis described herein is one in which there is the presence or absence of a 7-base deletion located at position 16 (according to SEQ ID NO: 33).

In another aspect of the invention there is provided a method for the diagnosis of IBD or determining susceptibility to develop IBD, which method comprises:
(i) obtaining a protein or nucleic acid containing sample from an individual;
(ii) detecting the presence or absence of a variant DLG5 on the basis of the presence of a polymorphic amino acid within the DLG5 protein, or a polymorphic base within the dlg5 gene sequence; and,
(iii) determining the status of the human by reference to the presence or absence of a polymorphism in DLG5.

In one embodiment the polymorphic amino acid is located at position 140, 231, 624, 1067, 1089 or 1481 according to SEQ ID NO: 2.

In a particular embodiment the polymorphism is selected from the group consisting of: Gln140Arg, Ser321Gly, Glu624Gln, Arg1067His, Pro1089Leu and Pro1481Gln according to SEQ ID NO: 2.

In one embodiment the polymorphic amino acid is located at position 30, 121, 514, 957, 979 or 1371 according to SEQ ID NO: 190.

In a particular embodiment the polymorphism is selected from the group consisting of: Gln30Arg, Ser121Gly, Glu514Gln, Arg957His, Pro979Leu and Pro1371Gin according to SEQ ID NO: 190.

The protein or nucleic acid containing test sample may conveniently be a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before use in the analysis of DLG5 variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. List 1 identifies a number of mutation detection techniques, some based on the polymerase chain reaction (PCR). These may be used in combination with a number of signal generation systems, a selection of which is listed in List 2. Further amplification techniques are listed in List 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

TABLE 1
Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| CMC | Chemical mismatch cleavage |
| Bp | base pair |
| COPS | Competitive oligonucleotide priming system |
| DGGE | Denaturing gradient gel electrophoresis |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SERRS | Surface enhanced raman resonance spectroscopy |
| SNP | Single nucleotide polymorphism |
| SSCP | Single-strand conformation polymorphism analysis |
| SSR | Self sustained replication |
| TGGE | Temperature gradient gel electrophoresis |
| 3′ UTR | 3′ untranslated region |

List 1—Mutation Detection Techniques

General: DNA sequencing, Sequencing by hybridisation

Scanning: PTT*, SSCP, DGGE, TGGE, Cleavase, Heteroduplex analysis, CMC, Enzymatic mismatch cleavage

*Note: not useful for detection of promoter polymorphisms.

Hybridisation Based: Solid phase hybridisation: Dot blots, MASDA, Reverse dot blots, Oligonucleotide arrays (DNA Chips)

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)

Extension Based: ARMS™-allele specific amplification (as described in European patent No. EP-B-332435 and U.S. Pat. No. 5,595,890), ALEX™—European Patent No. EP 332435 B1 (Zeneca Limited), COPS—Gibbs et al (1989), Nucleic Acids Research, 17, 2347.

Incorporation Based: Mini-sequencing, APEX

Restriction Enzyme Based: RFLP, Restriction site generating PCR

Ligation Based: OLA

Other: Invader assay, Hybridisation protection assay

List 2—Signal Generation or Detection Systems

Fluorescence: FRET, Fluorescence quenching, Fluorescence polarisation—United Kingdom Patent No. 2228998 (Zeneca Limited)

Other: Chemiluminescence, Electrochemiluminescence, Raman, Radioactivity, Colorimetric, Mass spectrometry, SERRS—WO 97/05280 (University of Strathclyde).

List 3—Further Amplification Methods

SSR, NASBA, LCR, SDA, b-DNA

Preferred mutation detection techniques include ARMS™-allele specific amplification, Taqman™, Mini sequencing, sequencing, RFLP, ALEX™, OLA, restriction site based PCR and FRET techniques.

Particularly preferred methods include ARMS™-allele specific amplification, OLA and RFLP based methods. ARMS™-allele specific amplification is an especially preferred method.

ARMS™-allele specific amplification (described in European patent No. EP-B-332435, U.S. Pat. No. 5,595,890 and Newton et al. (Nucleic Acids Research, Vol. 17, p.2503; 1989)), relies on the complementarity of the 3′ terminal nucleotide of the primer and its template. The 3′ terminal nucleotide of the primer being either complementary or non-complementary to the specific mutation, allele or polymorphism to be detected. There is a selective advantage for primer extension from the primer whose 3′ terminal nucleotide complements the base mutation, allele or polymorphism. Those primers which have a 3′ terminal mismatch with the template sequence severely inhibit or prevent enzymatic primer extension. Polymerase chain reaction or unidirectional primer extension reactions therefore result in product amplification when the 3′ terminal nucleotide of the primer complements that of the template, but not, or at least not efficiently, when the 3′ terminal nucleotide does not complement that of the template.

It will be appreciated that the test sample may equally be a nucleic acid sequence corresponding to the sequence in the test sample, that is to say that all or a part of the region in the sample nucleic acid may firstly be amplified using any convenient technique e.g. polymerase chain reaction (PCR), before analysis. The nucleic acid may be genomic DNA or fractionated or whole cell RNA. In one embodiment the RNA is whole cell RNA and is used directly as the template for labelling a first strand cDNA using random primers or poly A primers. The nucleic acid or protein in the test sample may be extracted from the sample according to standard methodologies (Sambrook et al. “Molecular Cloning—A Laboratory manual”, second edition. Cold Spring Harbor, N.Y. (1989)).

It will be apparent that the gene sequence disclosed for dlg5 (as depicted in SEQ ID NO: 1) is a representative sequence. In normal individuals there are two copies of each gene, a maternal and paternal copy, which will likely have some sequence differences, moreover within a population there will exist numerous allelic variants of the gene sequence, indeed the Examples identify numerous SNPs and other mutations within dlg5 gene that represent allelic variants within the population. It will be appreciated that the diagnostic methods and other aspects of this invention extend to the detection etc. of any of these sequence variants. Preferred sequence variants are those that possess at least 90% and preferably at least 95% sequence identity (nucleic acid or amino acid) to DLGS depicted in SEQ ID No. 1 or 2. Nucleic acid sequence identity can also be gauged by hybridisation studies whereby, under stringent hybridisation and wash conditions, only closely related sequences (for example, those with >90% identity) are capable of forming a hybridisation complex. In a further aspect, the diagnostic methods of the invention, are used to assess the predisposition and/or susceptibility of an individual to IBD, and the present invention may be used to recognise individuals who are particularly at risk from developing IBD conditions.

In a further aspect, the diagnostic methods of the invention are used in the development of new drug therapies, which selectively target one or more allelic variants identified herein. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes. The person of ordinary skill will be able to design and implement diagnostic procedures based on the detection of restriction fragment length polymorphism due to the loss or gain of one or more of the sites.

The invention further provides nucleotide sequence information, which can be used to design assays for detection of the polymorphisms of the invention.

The invention further provides nucleotide primers, which detect the polymorphisms of the invention.

The invention further provides nucleotide probes, which can detect the polymorphisms of the invention.

The amino acid sequence method for diagnosis is preferably one which is determined by immunological methods such as enzyme linked immunosorbent assay (ELISA).

The levels of the DLG5 can be assessed from relative amounts of mRNA, cDNA, genomic DNA or polypeptide sequence present in the test sample. Where RNA is used, it may be desired to convert the RNA to a complementary cDNA and during this process it may be desirable to incorporate a suitable detectable label into the cDNA.

In a preferred embodiment the method of the invention relies on detection of mRNA transcript levels. This involves assessment of the relative mRNA transcript levels of dlg5 in a sample, and comparison of sample data to control data. The gene transcript can be detected individually, or, is preferably detected amongst a panel of other disease-linked gene dlg5 from which a transcript profile can be generated. Levels of dlg5 mRNA in the test sample can be detected by any technique known in the art. These include Northern blot analysis, reverse transcriptase-PCR amplification (RT-PCR), microarray analysis and RNAse protection. In one embodiment, levels of dlg5 RNA in a sample can be measured in a Northern blot assay. Here, tissue RNA is fractionated by electrophoresis, fixed to a solid membrane support, such as nitrocellulose or nylon, and hybridised to a probe or probes capable of selectively hybridising with the dlg5 RNA to be detected. The actual levels may be quantitated by reference to one or more control housekeeping genes. Probes may be used singly or in combination. This may also provide information on the size of mRNA detected by the probe. Housekeeping genes are genes which are involved in the general metabolism or maintenance of the cell, and are considered to be expressed at a constant level irrespective of cell type, physiological state or stage in the cell cycle. Examples of suitable housekeeping genes are: beta actin, GAPDH, histone H3.3 or ribosomal protein L13 (Koehler et al., Quantitation of mRNA by Polymerase Chain Reaction. Springer-Verlag, Germany (1995)). To gauge relative expression levels, a control sample can be run alongside the test sample or, the test result/value can be compared to dlg5 expression levels expected in a normal or control tissue. These control values can be generated from prior test experiments using normal or control tissues, to generate mean or normal range values for dlg5.

In another embodiment, the dlg5 nucleic acid in a tissue sample is amplified and quantitatively assayed. The polymerase chain reaction (PCR) procedure can be used to amplify specific nucleic acid sequences through a series of iterative steps including denaturation, annealing of oligonucleotide primers (designed according to the sequence disclosed in SEQ ID NO. 1), and extension of the primers with DNA polymerase (see, for example, Mullis, et al., U.S. Pat. No. 4,683,202; Loh et al., Science 243:217 (1988)). In reverse transcriptase-PCR (RT-PCR) this procedure is preceded by a reverse transcription step to allow a large amplification of the number of copies of mRNA (Koehler et al., supra). Other known nucleic acid amplification procedures include transcription-based amplification systems (TAS) such as nucleic acid based sequence application (NASBA) and 3SR (Kwoh et al., Proc Natl. Acad Sci USA 86:1173 (1989), Gingeras et al., PCT application WO 88/10315), the ligase chain reaction (LCR, see European Application No. 320308), Strand Displacement Amplification (SDA), “race”, “one sided PCR” and others (Frohman, PCR Protocols: a Guide to Methods and Applications. Academic Press, NY (1990); Ohara et al., Proc Natl Acad Sci. USA 86:5673-5677 (1989)). Quantitation of RT-PCR products can be done while the reaction products are building up exponentially, and can generate diagnostically useful clinical data. In one embodiment, analysis is carried out by reference to one or more housekeeping genes which are also amplified by RT-PCR. Quantitation of RT-PCR product may be undertaken, for example, by gel electrophoresis visual inspection or image analysis, HPLC (Koehler et al., supra) or by use of fluorescent detection methods such as intercalation labelling, Taqman probe (Higuchi et al., Biotechnology 10:413-417 (1992)), Molecular Beacon (Piatek et al., Nature Biotechnol. 4:359-363 (1998)), primer or Scorpion primer (Whitcombe et al., Nature Biotech 17:804-807 (1999)); or other fluorescence detection method, relative to a control housekeeping gene or genes as discussed above.

Dlg5 RNA measurements can also be carried out on sinovial fluid, blood or serum samples. Preferably, the RNA is obtained from a peripheral blood sample. In the case of soluble RNA in the blood serum, the low abundance of mRNA expected would necessitate a sensitive test such as RT-PCR (Kopreski et al., Clin Cancer Res 5:1961-5 (1999)). A whole blood gradient may be performed to isolate nucleated cells and total RNA is extracted such as by the Rnazole B method (Tel-Test Inc., Friendsworth, Tex.) or by modification of methods known in the art such as described in Sambrook et al., (supra).

In a preferred embodiment of the invention, the diagnosis/detection method of the invention involves assessing dlg5 transcript levels using microarray analysis. Microarray technology makes it possible to simultaneously study the expression of many thousands of genes in a single experiment. Analysis of gene expression in human tissue (e.g. biopsy tissue) can assist in the diagnosis and prognosis of disease and the evaluation of risk for disease. A comparison of levels of expression of various genes from patients with defined pathological disease conditions with normal patients enables an expression profile, characteristic of disease, to be created.

Probes are made that selectively hybridise to the sequences of the target dlg5 gene in the test sample. These probes, perhaps together with other probes and control probes, are bound at discrete locations on a suitable support medium such as a nylon filter or microscope slide to form a transcript profiling array. The diagnostic method involves assessing the relative mRNA transcript level of dlg5 in a clinical sample. This can be done by radioactively labelling, or non-radioactively labelling the tissue mRNA, which can be optionally purified from total RNA, in any of a number of ways well known to the art (Sambrook et al., supra). The probes can be directed to any part or all of the target dlg5 mRNA.

In another embodiment of the invention, total dlg5 RNA or DNA is quantified and compared to levels in control tissue or expected levels from pre tested standards. DNA and/or RNA may be quantified using techniques well known in the art. Messenger RNA is often quantitated by reference to internal control mRNA levels within the sample, often relative to housekeeping genes (Koehler et al., supra).

In a preferred embodiment hybridisation signals generated are measured by computer software analysis of images on phosphorimage screens exposed to radioactively labelled tissue RNA hybridised to a microarray of probes on a solid support such as a nylon membrane. In another, quantities are measured by densitometry measurements of radiation-sensitive film (e.g. X-ray film), or estimated by visual means. In another embodiment quantities are measured by use of fluorescently labelled probe, which may be a mixture of biopsy and normal RNA differentially labelled with different fluorophores, allowing quantities of dlg5 mRNA to be expressed as a ratio versus the normal level. The solid support in this type of experiment is generally a glass microscope slide, and detection is by fluorescence microscopy and computer imaging.

The detection of specific interactions may be performed by detecting the positions where the labelled target sequences are attached to the array. Radiolabelled probes can be detected using conventional autoradiography techniques. Use of scanning autoradiography with a digitised scanner and suitable software for analysing the results is preferred. Where the label is a fluorescent label, the apparatus described, e.g. in International Publication No. WO 90/15070; U.S. Pat. No. 5, 143,854 or U.S. Pat. No. 5,744,305 may be advantageously applied. Indeed, most array formats use fluorescent readouts to detect labelled capture:target duplex formation. Laser confocal fluorescence microscopy is another technique routinely in use (Kozal et al., Nature Medicine 2:753-759 (1996)). Mass spectrometry may also be used to detect oligonucleotides bound to a DNA array (Little et al, Analytical Chemistry 69: 4540-4546, (1997)). Whatever the reporter system used, sophisticated gadgetry and software may be required in order to interpret large numbers of readouts into meaningful data (such as described, for example, in U.S. Pat. No. 5,800,992 or International Publication No. WO 90/04652).

In a preferred embodiment of the microarray test, the dlg5 RNA measurement is generated as a value relative to an internal standard (i.e. a housekeeping gene) known to be constant or relatively constant. The histone H3.3 and ribosomal protein L19 housekeeping genes have been shown to be cell-cycle independent and constitutively expressed in all tissues (Koehler et al., supra). For normalisation of data, several different housekeeping genes can be used to generate an average housekeeping measurement.

A microarray or RT-PCR test to detect IBD or susceptibility thereto can be used where tissue samples containing mRNA are available.

Samples for RNA extraction must be treated promptly to avoid RNA degradation (Sambrook et al., supra). This entails either prompt extraction using e.g. phenol-based reagents or snap freezing in e.g. liquid nitrogen. Samples can be stored at −70° C. or less until RNA can be extracted at a later date. Proprietary reagents are available which allow tissue or cells to be conveniently stored for several days at room temperature and up to several months at 4° C. (e.g. RNAlater, Ambion Inc., TX). Prior to extraction, methods such as grinding, blending or homogenisation are used to dissipate the tissue in a suitable extraction buffer. Typical protocols then use solvent extraction and selective precipitation techniques. In another embodiment oligonucleotide probe(s) capable of selectively hybridising to dlg5 nucleic acid, can be used to detect levels of dlg5 gene expression.

Convenient DNA sequences for use in the various aspects of the invention may be obtained using conventional molecular biology procedures, for example by probing a human genomic or cDNA library with one or more labelled oligonucleotide probes containing 10 or more contiguous nucleotides designed using the nucleotide sequences described here. Alternatively, pairs of oligonucleotides one of which is homologous to the sense strand and one to the antisense strand, designed using the nucleotide sequences described herein to flank a specific region of DNA may be used to amplify that DNA from a cDNA library.

Levels of dlg5gene expression can also be detected by screening for levels of polypeptide (DLG5 protein). For example, monoclonal antibodies immunoreactive with DLG5 protein can be used to screen a test sample. Such immunological assays can be done in any convenient format known in the art. These include Western blots, immunohistochemical assays and ELISA assays. Functional assays can also be used, such as protein binding determinations.

According to another aspect of the present invention, there is provided an allele specific primers or probes capable of detecting a polymorphism at position 16 in one or more of SEQ ID NOs: 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91 and 93. The person of ordinary skill in the art will be able to design suitable primers or probes using the sequence information provided herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Preferred primers for amplification are between 15 and 60 bases, more preferably between 17 and 35 bases in length. Probe sequences can be anything from about 25 nucleotides in length upwards. If the target sequence is a gene of 2 kb in size the probe sequence can be the complete gene sequence complement and thus may also be 2kb in size. Preferably, the probe sequence is a genomic, or more preferably a cDNA, fragment of the target sequence and may be between 50 and 2000 bases, preferably between 200 and 750 bases. It will be appreciated that multiple probes each capable of selectively hybridising to a different target sequence of the dlg5 nucleic acid, maybe across the complete length of the dlg5 gene sequence, may be prepared and used together in a diagnostic test. The primers or probes may be completely homologous to the target sequence or may contain one or more mismatches to assist specificity in binding to the correct template sequence. Any sequence, which is capable of selectively hybridising to the target sequence of interest, may be used as a suitable primer or probe sequence. It will also be appreciated that the probe or primer sequences must hybridise to the target template nucleic acid. If the target nucleic acid is double stranded (genomic or cDNA) then the probe or primer sequence can hybridise to the sense or antisense strand. If however the target is mRNA (single stranded sense strand) the primer/probe sequence will have to be the antisense complement.

An example of a suitable hybridisation solution when a nucleic acid is immobilised on a nylon membrane and the probe nucleic acid is greater than 500 bases or base pairs is: 6×SSC (saline sodium citrate), 0.5% SDS (sodium dodecyl sulphate), 100 μg/lml denatured, sonicated salmon sperm DNA. The hybridisation being performed at 68° C. for at least 1 hour and the filters then washed at 68° C. in 1×SSC, or for higher stringency, 0.1×SSC/0.1% SDS.

An example of a suitable hybridisation solution when a nucleic acid is immobilised on a nylon membrane and the probe is an oligonucleotide of between 12 and 50 bases is: 3M trimethylammonium chloride (TMACl), 0.01M sodium phosphate (pH 6.8), 1 mM EDTA (pH 7.6), 0.5% SDS,100 μg/ml denatured, sonicated salmon sperm DNA and 0.1 dried skimmed milk. The optimal hybridisation temperature (Tm) is usually chosen to be 5° C. below the Ti of the hybrid chain. Ti is the irreversible melting temperature of the hybrid formed between the probe and its target. If there are any mismatches between the probe and the target, the Tm will be lower. As a general guide, the recommended hybridisation temperature for 17-mers in 3M TMACl is 48-50° C.; for 19-mers, it is 55-57° C.; and for 20-mers, it is 58-66

According to another aspect of the present invention there is provided an allele-specific oligonucleotide probe capable of detecting a polymorphism in human nucleic acid corresponding to that at position 16 of any of SEQ ID NOs: 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91 and 93.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection. The sequences disclosed as SEQ ID Nos: 6-93, when in single stranded form, are representative examples of allele specific probes capable of detecting one or other of the polymorphic variants of dlg5. Each of these sequences is fully complementary to the native dlg5 gene and one or other of the particular allelic variants.

Primers or probes for use in any of the methods of the invention may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7 (1993); 1^{st} Edition. If required the primer(s) may be labelled to facilitate detection.

There are many conventional detectable labels such as radioisotopes, fluorescent labels, chemiluminescent compounds, labelled binding proteins, magnetic labels, spectroscopic markers and linked enzymes that might be used in conjunction with the primers or probes of the invention. One particular example well known in the art is end-labelling with ³²p. Fluorescent labels are preferred because they are less hazardous than radiolabels, they provide a strong signal with low background and various different fluorophors capable of absorbing light at different wavelengths and/or giving off different colour signals exist to enable comparative analysis in the same analysis. For example, fluorescein gives off a green colour, rhodamine gives off a red colour and both together give off a yellow colour.

The oligonucleotide primers and probes of the invention are particularly suitable for detecting the genotype of a particular SNP of dlg5.

The DLG5 protein of the invention and homologues or fragments thereof may be used to generate substances which selectively bind to it and in so doing regulate the activity of the protein. Such substances include, for example, antibodies, and the invention extends in particular to an antibody which is capable of binding to the protein shown in SEQ ID No:2. In particular the antibody may be a neutralising antibody.

As used herein the term antibody is to be understood to mean a whole antibody or a fragment thereof, for example a F(ab)2, Fab, FV, VH or VK fragment, a single chain antibody, a multimeric monospecific antibody or fragment thereof, or a bi- or multi-specific antibody or fragment thereof. Each of these types of antibody derivative and their acronyms are well known to the person skilled in the art.

In another preferred embodiment antibodies directed against DLG5 protein can be used, to detect, prognose, diagnose and stage IBD. Various histological staining methods known in the art, including immunochemical staining methods, may also be used. Silver stain is but one method of detecting DLG5 proteins. For other staining methods useful in the present invention see, for example, A Textbook of Histology, Eds. Bloom and Fawcett, W.B. Saunders Co., Philadelphia (1964).

According to a further aspect of the invention there is provided use of an antibody selective for DLG5 protein, in an assay to diagnose or prognose or monitor IBD.

The antibodies for use in this aspect of the invention can be prepared using the DLG5 protein/polypeptides.

Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)). The term antibody includes both monoclonal antibodies, which are a substantially homogeneous population, and polyclonal antibodies which are heterogeneous populations. The term also includes inter alia, humanised and chimeric antibodies. Monoclonal antibodies to specific antigens may be obtained by methods known to those skilled in the art, such as from hybridoma cells, phage display libraries or other methods. Monoclonal antibodies may be inter alia, human, rat or mouse derived. For the production of human monoclonal antibodies, hybridoma cells may be prepared by fusing spleen cells from an immunised animal, e.g. a mouse, with a tumour cell. Appropriately secreting hybridoma cells may thereafter be selected (Koehler & Milstein, Nature 256:495-497 (1975); Cole et al., “Monoclonal antibodies and Cancer Therapy”, Alan R Liss Inc, New York N.Y. pp 77-96 (1985)). Such antibodies may be of any immunoglobulin class including IgG, IgM, IgE, IgA, IgD and any subclass thereof. Polyclonal antibodies can be generated by immunisation of an animal (such as a mouse, rat, goat, horse, sheep etc) with an antigen, such as a DLG5 polypeptide.

The DLG5 polypeptide(s) can be prepared by various techniques known to the person skilled in the art. RNA transcripts can be used to prepare a polypeptide of the invention by in vitro translation techniques according to known methods (Sambrook et al. supra).

Alternatively, the DLG5 polypeptide(s) can be synthesised chemically. For example, by the Merryfield technique (J. Amer. Chem. Soc. 85:2149-2154, (1968)). Numerous automated polypeptide synthesisers, such as Applied Biosystems 431A Peptide Synthesizer also now exist. Alternatively, and preferably, the DLG5 polypeptide(s) are produced from a nucleotide sequence encoding the polypeptide using recombinant expression technology. A variety of expression vector/host systems may be used to express the dlg5 coding sequences. These include, but are not limited to microorganisms such as bacteria expressed with plasmids, cosmids or bacteriophage; yeasts transformed with expression vectors; insect cell systems transfected with baculovirus expression systems; plant cell systems transfected with plant virus expression systems, such as cauliflower mosaic virus; or mammalian cell systems (for example those transfected with adenoviral vectors); selection of the most appropriate system is a matter of choice. Preferably, the DLG5 protein is expressed in eukaryotic cells, especially mammalian, insect and yeast cells. Mammalian cells provide post-translational modifications to recombinant DLG5 protein, which include folding and/or phosphorylation.

Expression vectors usually include an origin of replication, a promoter, a translation initiation site, optionally a signal peptide, a polyadenylation site, and a transcription termination site. These vectors also usually contain one or more antibiotic resistance marker gene(s) for selection. As noted above, suitable expression vectors may be plasmids, cosmids or viruses such as phage or retroviruses. The coding sequence of the polypeptide is placed under the control of an appropriate promoter, control elements and transcription terminator so that the nucleic acid sequence encoding the polypeptide is transcribed into RNA in the host cell transformed or transfected by the expression vector construct. The coding sequence may or may not contain a signal peptide or leader sequence for secretion of the polypeptide out of the host cell. Expression and purification of the DLG5 polypeptide(s) can be easily performed using methods well known in the art (for example as described in Sambrook et al. supra).

The DLG5 polypeptide(s) so produced can then be used to inoculate animals, from which serum samples, containing the specific antibody against the introduced DLG5 protein/polypeptide, can later be obtained.

Rodent antibodies may be humanised using recombinant DNA technology according to techniques known in the art. Alternatively, chimeric antibodies, single chain antibodies, Fab fragments may also be developed against the polypeptides of the invention (Huse et al., Science 256:1275-1281 (1989)), using skills known in the art. Antibodies so produced have a number of uses, which will be evident to the molecular biologist or immunologist skilled in the art. Such uses include, but are not limited to, monitoring enzyme expression, development of assays to measure enzyme activity and use as a therapeutic agent. Enzyme linked immunosorbant assays (ELISAs) are well known in the art and would be particularly suitable for detecting the DLG5 protein or polypeptide fragments thereof in a test sample.

The DLG5 specific antibodies can be used in an ELISA assay to detect DLG5 protein in body fluids or by immunohistochemistry or other means. In addition, an antibody could be used individually or as part of a panel of antibodies, together with a control antibody, which reacts to a common protein, on a dipstick or similar diagnostic device.

All the essential materials and reagents required for detecting DLG5 in a test sample may be assembled together in a kit. Such a kit may comprise one or more diagnostic cDNA probes or oligonucleotide primers together with control probes/primers. The kit may contain probes immobilised on a microarray substrate such as a filter membrane or silicon-based substrate. The kit may also comprise samples of total RNA derived from tissues of various physiological states, such as normal, BPH, confined tumour and metastatic tumour, for example, to be used as controls. The kit may also comprise appropriate packaging and instructions for use in the methods of the invention.

According to another aspect of the present invention there is provided a diagnostic kit for diagnosing or prognosing or monitoring IBD comprising, one or more diagnostic probe(s) and/or diagnostic primer(s) and/or antibodies capable of selectively hybridising or binding to DLG5.

It will be appreciated that the term “diagnostic kit” is not intended to limit the kit to diagnostic use only, it also encompasses other uses such as in prognostic, stage monitoring and therapeutic efficacy studies.

In a preferred embodiment, the diagnostic (detection) probes are provided on a microarray.

Such kits may further comprise appropriate buffer(s) and/or polymerase(s) such as thermostable polymerases, for example taq polymerase. They may also comprise companion/constant primers and/or control primers or probes. A companion/constant primer is one that is part of the pair of primers used to perform PCR. Such primer usually complements the template strand precisely. The kit may also contain control normal RNA labelled with one fluorophore (E.g. Cy5). In use, patient RNA derived from biopsy or body fluids or cells can be labelled with another fluorophore (e.g. Cy3), the RNAs could then be mixed and hybridised to the array. Instrumentation to detect fluorescence ratio e.g. of. Cy3:Cy5 are available and could be used to detect DLG5 over-expression.

In another embodiment the kit comprises one or more specific probes suitable for hybridisation to mRNA in tissue sections in situ. The kit may also contain hybridisation buffer and detection reagents for colourimetric or fluorescence microscopy detection. In another embodiment the kit comprises a set of specific oligonucleotide primers, optionally labelled, for quantitation by RT-PCR of dlg5mRNA. These primers may be Scorpion primers (Whitcombe et al., Nature Biotechnol. 17:804-807, 1999) allowing accurate quantitation of specific PCR product. Alternatively, Taqman or Molecular Beacon probes may be provided in the kit for this purpose. One form of the kit would be a microtitre plate containing specific reagents in several wells, to which aliquots of extracted RNA could be pipetted. The microtitre plate could be thermocycled on a suitable machine, which could also be capable of reading fluorescence emissions from plate wells (e.g. Perkin Elmer 7700).

In another embodiment the kit comprises one or more antibodies specific for the DLG5 protein for use in immunohistochemical analysis.

In another embodiment the kit is an ELISA kit comprising one or more antibodies specific for the DLG5 protein identified herein.

In another aspect of the invention there is provided a method for treating a patient suffering from IBD comprising administering to the patient an effective amount of an antibody specific for DLG5.

According to another aspect of the invention, the dlg5 gene may be used in gene therapy, for example where it is desired to modify the production of the protein in vivo, and the invention extends to such uses.

Knowledge of the gene according to the invention also provides the ability to regulate its expression in vivo by for example the use of antisense DNA or RNA. One therapeutic means of inhibiting or dampening the expression levels of a particular gene (for example dlg5 identified herein) is to use antisense therapy. Antisense therapy utilises antisense nucleic acid molecules that are synthetic segments of DNA or RNA (“oligonucleotides”), designed to mirror specific mRNA sequences and block protein production. Once formed, the mRNA binds to a ribosome, the cell's protein production “factory” which effectively reads the RNA sequence and manufactures the specific protein molecule dictated by the gene. If an antisense molecule is delivered to the cell (for example as native oligonucleotide or via a suitable antisense expression vector), it binds to the messenger RNA because its sequence is designed to be a complement of the target sequence of bases. Once the two strands bind, the mRNA can no longer dictate the manufacture of the encoded protein by the ribosome and is rapidly broken down by the cell's enzymes, thereby freeing the antisense oligonucleotide to seek and disable another identical messenger strand of mRNA.

Thus, according to another aspect of the invention there is provided a method for treating a patient suffering from IBD comprising administering to said patient an effective amount of an antisense molecule capable of binding to the mRNA of the dlg5 gene, and inhibiting expression of the protein product of the dlg5 gene.

Complete inhibition of protein production is not essential, indeed may be detrimental. It is likely that inhibition to a state similar to that in normal tissues would be desired.

This aspect of antisense therapy is particularly applicable if the IBD disorder is a direct cause of over-expression of the dlg5 gene in question, although it is equally applicable if said dlg5 gene indirectly cause the IBD disorder. With knowledge of the dlg5 gene and mRNA sequence, the person skilled in the art is able to design suitable antisense nucleic acid therapeutic molecules and administer them as required.

Antisense oligonucleotide molecules with therapeutic potential can be determined experimentally using well established techniques. To enable methods of down-regulating expression of the dlg5 gene of the present invention in mammalian cells, an example antisense expression construct can be readily constructed for instance using the pREP10 vector (Invitrogen Corporation). Transcripts are expected to inhibit translation of the gene in cells transfected with this type of construct. Antisense transcripts are effective for inhibiting translation of the native gene transcript, and capable of inducing the effects (e.g., regulation of tissue physiology) herein described. Oligonucleotides which are complementary to and hybridisable with any portion of dlg5 gene mRNA are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Expression vectors containing random oligonucleotide sequences derived from the dlg5 gene sequence are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material. Antisense molecules can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

As noted above, antisense nucleic acid molecules may also be provided as RNAs, as some stable forms of RNA are now known in the art with a long half-life that may be administered directly, without the use of a vector. In addition, DNA constructs may be delivered to cells by liposomes, receptor mediated transfection and other methods known to the art.

The antisense DNA or RNA for co-operation with the gene in SEQ ID No:1 can be produced using conventional means, by standard molecular biology and/or by chemical synthesis as described above. If desired, the antisense DNA or antisense RNA may be chemically modified so as to prevent degradation in vivo or to facilitate passage through a cell membrane and/or a substance capable of inactivating mRNA, for example ribozyme, may be linked thereto and the invention extends to such constructs.

The antisense DNA or antisense RNA may be of use in the treatment of diseases or disorders in humans in which the over- or under-regulated production of the dlg5 gene product has been implicated.

Alternatively, ribozyme molecules may be designed to cleave and destroy the dlg5 mRNA in vivo. Ribozymes are RNA molecules that possess highly specific endoribonuclease activity. Hammerhead ribozymes comprise a hybridising region, which is complementary in nucleotide sequence to at least part of the target RNA, and a catalytic region, which is adapted to recognise and cleave the target RNA. The hybridising region preferably contains at least 9 nucleotides. The design, construction and use of such ribozymes is well known in the art and is more fully described in Haselhoff and Gerlach, (Nature. 334:585-591, 1988). In another alternative oligonucleotides designed to hybridise to the 5′-region of the dlg5 gene so as to form triple helix structures may be used to block or reduce transcription of the dlg5 gene. In another alternative, RNA interference (RNAi) oligonucleotides or short (18-25 bp) RNAi dlg5 sequences cloned into plasmid vectors are designed to introduce double stranded RNA into mammalian cells to inhibit and/or result in the degradation of dlg5 messenger RNA. Dlg5 RNAi molecules may begin adenine/adenine (AA) or at least (any base-A,U,C or G)A . . . and may comprise of 18 or 19 or 20 or 21 or 22 or 23, or 24 or 25 base pair double stranded RNA molecules with the preferred length being 21 base pairs and be specific to individual dlg5 sequences with 2 nucleotide 3′ overhangs or hairpin forming 45-50 mer RNA molecules. The design, construction and use of such small inhibitory RNA molecules is well known in the art and is more fully described in the following: Elbashir et al., (Nature. 411(6836):494-498, 2001); Elbashir et al., (Genes & Dev. 15:188-200, 2001); Harborth, J. et al. (J. Cell Science 114:4557-4565, 2001); Masters et al. (Proc. Natl. Acad. Sci. USA 98:8012-8017, 2001); and, Tuschl et al., (Genes & Dev. 13:3191-3197, 1999).

Pathway mapping may be used to determine each protein in the cell with which the DLG5 protein interacts and, in turn, the proteins with which each of these proteins interacts also. In this way it is possible to identify the specific critical signaling pathway which links the disease stimulus to the cell's response thereby enabling the identification of new potential targets for therapy intervention.

According to a further aspect of the invention there is provided the use of the dlg5 gene or a fragment thereof in research to identify further gene targets implicated in IBD.

In another aspect of the invention, the single nucleotide polymorphisms of this invention may be used as genetic markers for this region in linkage studies.

Further features of the invention include:

A method of treatment of a patient suffering from inflammatory bowel disease, comprising administration to the patient of a compound capable of reducing the transcription or activity of dlg5 gene products.

A method of treatment of a patient suffering from IBD, comprising administration to the patient an inhibitory nucleic acid molecule targeted against the mRNA of dlg5.

Use of an inhibitory nucleic acid molecule against dlg5 or an antibody directed against DLG5 proteins, in the manufacture of a medicament for treating IBD.

As used herein, the term “inhibitory nucleic acid molecule” refers to molecules selected from the group consisting of: antisense, ribozyme, triple helix aptemer and RNAi molecules.

According to a further aspect of the invention there is provided a method of treating a human in need of treatment with a small molecule drug acting on the DLG5 protein or a drug comprising an inhibitory nucleic acid molecule acting against dlg5, in which the method comprises:
i) detection of a polymorphism in the dlg5 gene in the human, which diagnosis preferably comprises determining the nucleotide present within human dlg5 gene that occurs at position 16 in the nucleic acid that corresponds to any of SEQ ID Nos: 7, 9, 11, 13, 15, 17, 19, 21, 23, 25, 27, 29, 31, 33, 35, 37, 39, 41, 43, 45, 47, 49, 51, 53, 55, 57, 59, 61, 63, 65,.67, 69, 71, 73, 75, 77, 79, 81, 83, 85, 87, 89, 91 and 93;
ii) determining the status of the human by reference to polymorphism in the dlg5 gene; and,
iii) administering an effective amount of the drug.

According to a further aspect of the invention there is provided a method of treating a human in need of treatment with a small molecule drug acting on the DLG5 protein or a drug comprising an inhibitory nucleic acid molecule acting against the dlg5 mRNA, in which the method comprises:
i) measuring the level of the dlg5 mRNA in a tissue sample obtained from the human and,
ii) determining the status of the human by reference to normal levels of the dlg5 mRNA; and,
iii) administering an effective amount of the drug.

According to a further aspect of the invention there is provided a method of treating a human in need of treatment with a small molecule drug acting on the DLG5 protein or a drug comprising an inhibitory nucleic acid molecule acting against the dlg5 mRNA, in which the method comprises:
i) measuring the level of the dlg5 protein in a tissue sample obtained from the human and,
ii) determining the status of the human by reference to normal levels of the DLG5 protein; and,
iii) administering an effective amount of the drug.

According to a further aspect of the invention there is provided a method of treating a human in need of treatment with a small molecule drug acting on the DLG5 protein or a drug comprising an inhibitory nucleic acid molecule acting against the dlg5 mRNA, in which the method comprises:
i) detection of a polymorphism in the DLG5 protein in the human, which diagnosis preferably comprises determining the amino acid at any one of positions 140, 231, 624, 1067, 1089 or 1481 of the DLG5 protein sequence shown in SEQ ID NO: 2;
ii) determining the status of the human by reference to polymorphism in the DLG5 protein; and,
iii) administering an effective amount of the drug.

According to a further aspect of the invention there is provided a method of treating a human in need of treatment with a small molecule drug acting on the DLG5 protein or a drug comprising an inhibitory nucleic acid molecule acting against the dlg5 mRNA, in which the method comprises:
i) detection of a polymorphism in the DLG5 protein in the human, which diagnosis preferably comprises determining the amino acid at any one of positions 30, 121, 514, 957, 979 and 1371 of the DLG5 protein sequence shown in SEQ ID NO: 190;
ii) determining the status of the human by reference to polymorphism in the DLG5 protein; and,
iii) administering an effective amount of the drug.

A method of treatment of a patient suffering from IBD, comprising administration to the patient of a compound capable of reducing the transcription or expression of dlg5.

A method of treatment of a patient suffering from IBD, comprising administration to the patient an inhibitory nucleic acid molecule targeted against the mRNA of dlg5.

Use of an inhibitory nucleic acid molecule or an antibody directed against dlg5, in the manufacture of a medicament for treating IBD.

The invention will be further described by way of the following non-limiting examples and figures in which,

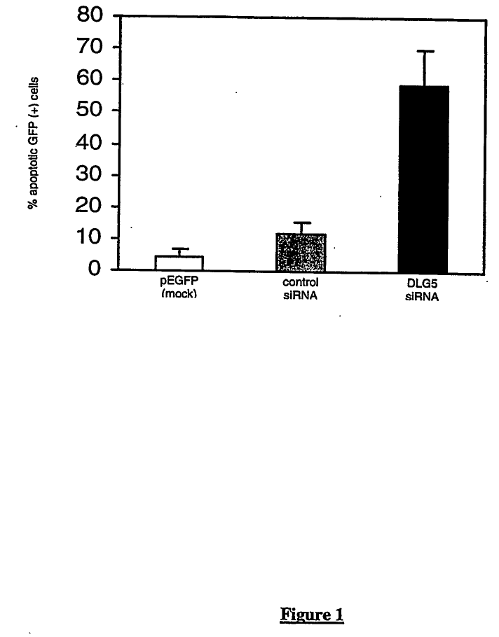

FIG. 1—a—represents mRNA levels of DLG5 and the housekeeping protein b-actin; b—activation of the key apoptotic effector caspase-3 and cleavage of its substrate poly(ADP-ribose)polymerase-1 (PARP-1) in cells treated with the specific siRNA directed against DLG5; c—Cells transfected with DLG5 siRNA showed a 48% increase of apoptosis (determined by fragmented nuclei stained with DAPI) as compared to cells treated with the scrambled control siRNA; data from a representative experiment are shown.

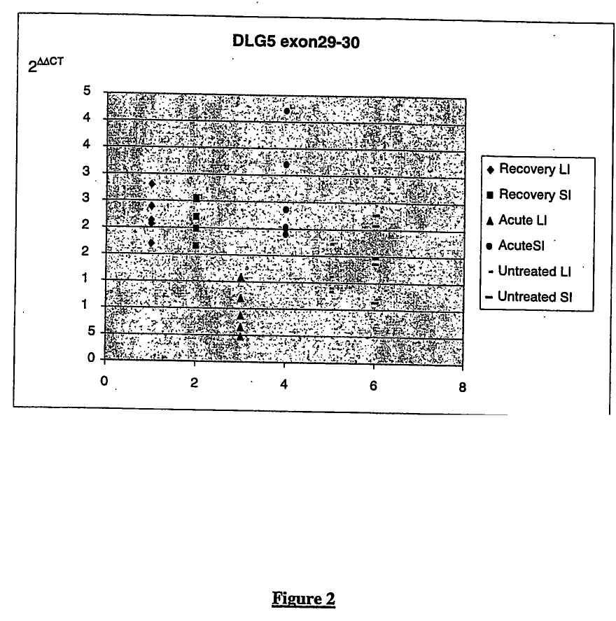

FIG. 2—TaqMan analyses of DLG5 expression in DSS colitis model of C57B/6 mice. Relative expression is indicated as 2^{ΔΔCT} of DLG5. LI (large intestine), SI (small intestine).

### EXAMPLE 1

Identification of dlg5 as a gene associated with IBD.

A genome-wide linkage scan involving 268 families (356 affected sibling pairs) of European descent was carried out to identify a susceptibility locus for IBD. Subsequently, a hierarchical linkage disequilibrium study was employed to search for the causal variant(s) within a broad 40 cM pericentromeric interval on chromosome 10, identified from an initial linkage scan. This endeavour was started with a fine mapping experiment involving 523 affected sibling pairs and 16 microsatellite markers at an average distance of 2 cM. The fine mapping experiment confirmed the initial linkage study, revealing a linkage peak at 10q, extending from D10S201-D10S192, with a maximum MLS score 1.6 at D10S2470. Linkage mapping was followed by transmission disequilibrium testing (TdT) using the algorithm implemented in the GENEHUNTER software package (Daly M J et al., Am J Hum Genet, Suppl 63:A286, 1998) to further narrow the region containing the susceptibility gene. This analysis tests for association of a given marker with the disease phenotype in the presence of linkage, and represents the most powerful and robust test for association, while omitting the risk of false positives due to stratification bias. The TdT performed on a single trio (one of the affected sibpairs and its parents) from each of the families showed a significant single point association with the disease phenotype at D10S547 (p<0.001) and D10S201 (p<0.01). This led to the following strategy: the genomic region underlying the linkage peaks on 10p14-10p13 (extending from 8 Mb-13 Mb) and 10q22-10q23 (77 Mb-82 Mb) were genotyped with 107 (SNPs) in 200 German IBD families (from the original linkage set) plus an additional 555 German Trios (368 CD, 187 UC), (a trio being a DNA sample from a IBD affected child plus DNA samples from its mother and father respectively) and 548 German control individuals (non IBD affected individuals). Several SNPs showed significant association with CD and IBD, respectively, by TdT in single trios extracted from the families and the trios, further confirming the presence of linkage disequilibrium with the susceptibility locus over the investigated region. It was therefore decided to perform additional association studies with SNPs at an average density of 75-120 kB to further narrow the region harbouring the susceptibility gene. Upon analysis of the high-density SNP panel, the lead region on 10q was scanned for nucleotide sequence variants as a highly significant single point association of the SNP marker TSC0376484 located at 78.5 Mb to IBD was found with a χ2=11.5, p=0.00067. TdT haplotype analyses further strengthened the association lead at TSC0376484 resulting in significant 2-marker and 3-marker haplotypes with the neighbouring SNP markers TSC0005010 and TSC0000361. A maximum χ2=15.44, p=0.00008 was observed for the 2-marker haplotype TSC0376484-TSC0000361 spanning a physical distance of approximately 150 kb. The positive SNP was located adjacent to the dlg5 gene. To verify and further clarify the role of dlg5 in IBD, 6 gene-based markers for dlg5 were genotyped. The analysis of these markers also involving testing for haplotype blocks, as described by Daly et al. (Nature Genet. 29:229-232, 2001), clearly showed that the association signal is entirely confined to the dlg5 gene with a total of 17 markers with a positive association with IBD (Table 5), all of which are located on a common underlying haplotype. Identical genetic association studies were also carried out on an adjacent gene. These proved negative, demonstrating that dlg5 is the sole candidate for the susceptibility locus for IBD on 10q22. In depth re-sequencing for all 32 exons and the exon-intron boundaries was performed in 47 individuals with proven diagnosis for IBD. Nucleotide sequencing was performed according to standard protocols, and the primers used are listed in Table 4. The DNA sequencing and analysis identified 20 novel nucleotide sequence variations (in addition to various publicly available SNPs) located in the dlg5 gene, 3 of which lead to an amino acid change of the protein, and further a 7 bp-deletion in the intron flanking exon 13. The genotype-related risk (GRR) (Risch&Merikangas, Science, 273(5281):1516-7, 1996) is estimated to be 1.5-2.5 based on the TDT results.

### EXAMPLE 2

### Cloning and Sequence Analysis of dlg5

A Genomic DNA Contig of 415779 bp was Constructed by Assembly of BAC Clones AL391421, AL450306 and AL731556

Using the database entry for dlg5 cDNA (EMBL accession number AF352034), all exons but the 5′ UTR were mapped to the human genomic sequences covered by AL391421 or AL4503306. The first 94 bases of AF352034 do not map to this region or any other region in the genomic contig around the gene. Since this sequence showed similarities to multiple regions within the human genome it was considered as an artifact derived from repetitive sequence. A database BLAST search using sequence from exon 2 was used to identify two pig cDNAs containing novel 5′ sequence (EMBL accession numbers BM 484383 and BI402246). This sequence was found to match the human genomic contig and was also flanked by a conserved splice site on the 3′ end and is therefore considered as the true 5′ exon of the human dlg5. Table 2 shows intron/exon border sequence information of the DLG5 gene. SEQ ID NO: 1 shows the sequence of dlg5 including the novel 5′ sequence. SEQ ID NO: 2 shows the predicted amino acid sequence of dlg5.

In depth re-sequencing for all 32 exons and the exon-intron boundaries was performed in 47 individuals with IBD. Sequences were aligned for SNP detection and in addition to detection of SNPs present in public databases, the analysis resulted in the identification of 20 novel SNPs, four of which lead to an amino acid change of the protein, as well as two novel genomic short deletions, one a 7 bp-deletion in the intron flanking exon 13, potentially influencing the splicing of the gene. The genotype-related risk (GRR) is estimated to be 1.5-2.5 based on the TDT results. Table 3 lists the identified SNPs and adjacent sequence as well as the allelic versions of the SNPs. The Table also includes positions of typed SNPs from public databases. Table 4 shows the primer sequences used to PCR amplify the SNP containing regions of dlg5.

TABLE 2

| | | size |
|---|---|---|
| EXON | SEQUENCE | (bp) |
| exon | ......GAAGGCGCGGgtgag | (>166) |
| 01 | | |
| exon | cccagGTTCTACCTA......AGCAGTGTGGgtgag | 69 |
| 02 | | |
| exon | cacagGCACTACCGG......TTGACAAGAGgtagt | 163 |
| 03 | | |
| exon | gccagGCCCTACCAC......ACTTCTACCAgtgag | 144 |
| 04 | | |
| exon | ttcagCACACTCCAC......GCAGCAGCAGgtagg | 184 |
| 05 | | |
| exon | tccagGTGTTGAAGC......CCCTGAGGAGgtagg | 260 |
| 06 | | |
| exon | gccagGTTTGAGGCG......GCTGCGGCAGgtagg | 313 |
| 07 | | |
| exon | accagATCAAAGACA......ACAGCATCCGgtatg | 185 |
| 08 | | |
| exon | accagGACACTGTGT......AGGAGCTCAAgtagg | 126 |
| 09 | | |
| exon | cacagGGAACAGATG......GAGGGAGACGgtaag | 133 |
| 10 | | |
| exon | tgcagGAGGATATTG......GCCGCTTAAGgtaag | 128 |
| 11 | | |
| exon | ctcagGGTCAATGAC......GGACAGAAAGgtagc | 176 |
| 12 | | |
| exon | tgcagACAGTGGCAT......GATCGTTGCGgtaag | 104 |
| 13 | | |
| exon | tctagATCAATGGCA......CCTCCTGAAGgtaag | 93 |
| 14 | | |
| exon | cctagGTATTCCCTC.....GTTCCTGGAGgtata | 1020 |
| 15 | | |
| exon | tgcagGAACAGAAGT......CCGTCTGTGGgtgag | 124 |
| 16 | | |
| exon | tctagGCACTGTTCC......TGCATCCCAGgtatg | 145 |
| 17 | | |
| exon | cgcagTGTCCAGCAC......GCCCGCCTGGgtaac | 113 |
| 18 | | |
| exon | aatagGTTCTTCGAG......TCCGAGAGAGgtaag | 90 |
| 19 | | |
| exon | tgcagGTTCAGTGTC......GAAAGGACAGgtgag | 151 |
| 20 | | |
| exon | cttagGCCTTATGTG......GTTACTGGAGgtgag | 163 |
| 21 | | |
| exon | cccagTTCAACGGCA......CCCGGTCCAGgtgag | 134 |
| 22 | | |
| exon | tccagCTCACACCTG......GCAGCTCCAGgtcag | 141 |
| 23 | | |
| exon | gaaagGATTGCGGGA......CATCCTGGAGgtgag | 184 |
| 24 | | |
| exon | ggcagTATGGCAGCC......TCTACATCAGgtacc | 149 |
| 25 | | |
| exon | tccagGGCCCTGTAC......GCAAATATGTgtaag | 171 |
| 26 | | |
| exon | cacagGATGGACCAA......CTCTTTGAAGgcaag | 197 |
| 27 | | |
| exon | ctcagATTCGGTGAG......TGTCCCCTTGgtaag | 144 |
| 28 | | |
| exon | tgcagAGGTGATGAA......CACAGAAAAGgtacc | 128 |
| 29 | | |
| exon | cccagAACCGACACT......AGCACATCAAgtagg | 110 |
| 30 | | |
| exon | cacagGGAGCAGAGA......TACTTCACAGgtagg | 110 |
| 31 | | |
| exon | tgcagGGGTCATCCA......... | (>1749) |
| 32 | | |

Table 2 shows exon/intron borders for all exons of the dlg5 gene. The first and last 10 bp of each exon (capital letters) together with 5 bp of surrounding introns (lowercase letters) are indicated. Also, the total size of each exon is indicated. All sequences can be identified within human BAC clones with accession number AL391421 and AL450306.

TABLE 3

| | | | |
|---|---|---|---|
| FEATURE | COMMENTS | SEQUENCE | SEQ ID |

| | | | |
|---|---|---|---|
| tsc0000361, | SNP, G to A, | gctcggtggcagcgaGtgagaggagctcagt | 6 |
| allele 1 | intron | | |
| tsc0000361, | | gctcggtggcagcgaAtgagaggagctcagt | 7 |
| allele 2 | | | |
| DLGe2, | SNP, C to T, | tggtctcccctctttCcccaggttctaccta | 8 |
| allele 1 | 5′ of exon 2 | | |
| DLGe2, | | tggtctcccctctttTcccaggttctaccta | 9 |
| allele 2 | | | |
| rs1248655, | SNP, C to T, | gtgggtgagtaccacCgtctggggaggacac | 10 |
| allele 1 | 3′ of exon 2 | | |
| rs1248655, | | gtgggtgagtaccacTgtctggggaggacac | 11 |
| allele 2 | | | |
| rs1248696, | SNP, A to G, | ccctcctcactgaccAgcaagtgaatgagaa | 12 |
| allele 1 | within exon 3a, | | |
| rs1248696, | non-synonymous | ccctcctcactgaccGgcaagtgaatgagaa | 13 |
| allele 2 | | | |
| DLGe3, | SNP, G to C, | ccgcaagcgcctggcCtttgctacgcatggc | 14 |
| allele 1 | within exon 3, | | |
| DLGe3, | synonymous | ccgcaagcgcctggcGtttgctacgcatggc | 15 |
| allele 2 | | | |
| rs1248695, | SNP, C to T, | ctgagtgtcccctttCccccacctcatgtcc | 16 |
| allele 1 | 3′ of exon 3 | | |
| rs1248695, | | ctgagtgtcccctttTccccacctcatgtcc | 17 |
| allele 2 | | | |
| DLGe5A, | SNP, A to G, | ttcagcacactccacAgccggctcctgagtg | 18 |
| allele 1 | within exon 5, | | |
| DLGe5A, | non-synonymous | ttcagcacactccacGgccggctcctgagtg | 19 |
| allele 2 | | | |
| DLGe5B, | SNP, C to A, | cccagcccctggagaCtggccatttctccca | 20 |
| allele 1: | 3′ of exon 5 | | |
| DLGe5B, | | cccagcccctggagaAtggccatttctccca | 21 |
| allele 2: | | | |
| rs1248680, | SNP, A to C, | ggcagccacccacctActcagatccagccta | 22 |
| allele 1 | intron | | |
| rs1248680, | | ggcagccacccacctCctcagatccagccta | 23 |
| allele 2 | | | |
| DLGe7, | SNP, G to A, | gagaaccacgcaggtGaagacagcaaaggag | 24 |
| allele 1 | within exon 7, | | |
| DLGe7, | synonymous | gagaaccacgcaggtAaagacagcaaaggag | 25 |
| allele 2 | | | |
| rs1270912, | SNP, T to C, | ctcagctgtggtggaTagactggacagtgcc | 26 |
| allele 1 | intron | | |
| rs1270912, | | ctcagctgtggtggaCagactggacagtgcc | 27 |
| allele 2 | | | |
| DLGe10, | SNP, G to C, | gaagttgtagagttcGagagggagacggtaa | 28 |
| allele 1 | within exon 10, | | |
| DLGe10, | non-synonymous | gaagttgtagagttcCagagggagacggtaa | 29 |
| allele 2 | | | |
| DLGe13A, | SNP, C to T, | ggagtgtatgctgcCgctgtgctgcctgga | 30 |
| allele 1 | within exon 13, | | |
| DLGe13A, | synonymous | ggagtgtatgctgcTgctgtgctgcctgga | 31 |
| allele 2 | | | |
| DLGe13B, | deletion | gcggtaagtctcaagGCTGGAGccagggt | 32 |
| allele 1 | GCTGGAG 3′ of | catctgcc | |
| DLGe13B, | exon 13 | gcggtaagtctcaagccagggtcatctgcc | 33 |
| allele 2 | | | |
| DLGe14A, | SNP, G to A, | ggtaggcctgaggccGctctgcctgtggcct | 34 |
| allele 1 | 5′ of exon 14 | | |
| DLGe14A, | | ggtaggcctgaggccActctgcctgtggcct | 35 |
| allele 2 | | | |
| rs1248629, | SNP, C to G, | tgaatctctgctgcgCagctgccaggactcc | 36 |
| allele 1 | within exon 14, | | |
| rs1248629, | synonymous | tgaatctctgctgcgGagctgccaggactcc | 37 |
| allele 2 | | | |
| DLGe14B, | SNP, G to A, | catgctactccttggGgtcacaggatccttg | 38 |
| allele 1 | 3′ of exon 14 | | |
| DLGe14B, | | catgctactccttggAgtcacaggatccttg | 39 |
| allele 2 | | | |
| DLGe15A, | SNP, C to T, | cggggagcccatgcaCgcatcaccccctcgc | 40 |
| allele 1 | within exon 15, | | |
| DLGe15A, | synonymous | cggggagcccatgcaTgcatcaccccctcgc | 41 |
| allele 2 | | | |
| DLGe15B, | SNP, G to A, | acgcatcaccccctcGcaaggccagggtccg | 42 |
| allele 1 | within exon 15, | | |
| DLGe15B, | non-synonymous | acgcatcaccccctcAcaaggccagggtccg | 43 |
| allele 2 | | | |
| DLGe15C, | SNP, C to T, | actcctcccacctgcCggccaagaaatcctg | 44 |
| allele 1 | within exon 15, | | |
| DLGe15C, | non-synonymous | actcctcccacctgcTggccaagaaatcctg | 45 |
| allele 2 | | | |
| rs2289308, | SNP, C to T, | ctgggtcctttggggCgtcttttctcaccaa | 46 |
| allele 1 | 5′ of exon 17 | | |
| rs2289308, | | ctgggtcctttggggTgtcttttctcaccaa | 47 |
| allele 2 | | | |
| rs1248634, | SNP, C to T, | aagcccatttctaggCactgttccccggagt | 48 |
| allele 1 | within exon 17, | | |
| rs1248634, | synonymous | aagcccatttctaggTactgttccccggagt | 49 |
| allele 2 | | | |
| rs1248635, | SNP, C to T, | tacgcttctctgtacCccagctgcccaagcc | 50 |
| allele 2 | 3′ of exon 17 | | |
| rs1248635, | | tacgcttctctgtacTccagctgcccaagcc | 51 |
| allele 4 | | | |
| DLGe18, | SNP, G to C, | tcctgggactgagctGatttctctactggga | 52 |
| allele 1 | 3′ of exon 18 | | |
| DLGe18, | | tcctgggactgagctCatttctctactggga | 53 |
| allele 2 | | | |
| DLGe19, | SNP, C to T, | gagtgtcgtgggctcCgagagaggtaaggac | 54 |
| allele 1 | within exon 19, | | |
| DLGe19, | synonymous | gagtgtcgtgggctcTgagagaggtaaggac | 55 |
| allele 2 | | | |
| rs1248625, | SNP, G to C, | tcctagggaacagcaGtgctcccaagtcccc | 56 |
| allele 1 | 3′ of exon 21 | | |
| rs1248625, | | tcctagggaacagcaCtgctcccaagtcccc | 57 |
| allele 2 | | | |
| DLGe23, | SNP, C to T, | acacctggaccctgcCggtacccactccact | 58 |
| allele 1 | within exon 23, | | |
| DLGe23, | synonymous | acacctggaccctgcTggtacccactccact | 59 |
| allele 2 | | | |
| rs2289310, | SNP, C to A, | ggcctagcaccccccCagccaagcagagcag | 60 |
| allele 1 | within exon 23, | | |
| rs2289310, | non-synonymous | ggcctagcaccccccAagccaagcagagcag | 61 |
| allele 2 | | | |
| rs1261990, | SNP, T to A, | aggagggatgttgaaTttctgccgtatggtc | 62 |
| allele 1 | 5′ of exon 25 | | |
| rs1261990, | | aggagggatgttgaaAttctgccgtatggtc | 63 |
| allele 2 | | | |
| DLGe25A, | SNP, C to G, | gccgtatggtcagcaCtggcccctctcgggt | 64 |
| allele 1 | 5′ of exon 25 | | |
| DLGe25A, | | gccgtatggtcagcaGtggcccctctcgggt | 65 |
| allele 2 | | | |
| DLGe25B, | SNP, G to A, | gcactggcccctctcGggtgcccaagctgcc | 66 |
| allele 1 | 5′ of exon 25 | | |
| DLGe25B, | | gcactggcccctctcAggtgcccaagctgcc | 67 |
| allele 2 | | | |
| rs1058198, | SNP, C to T, | gagctttaagaaggaCgacatcctctacgtg | 68 |
| allele 1 | within exon 26, | | |
| rs1058198, | synonymous | gagctttaagaaggaTgacatcctctacgtg | 69 |
| allele 2 | | | |
| DLGe26, | deletion of A, | ggggtggggtggggcAggggtcgccgagggc | 70 |
| allele 1 | 3′ of exon 26 | | |
| DLGe26, | | ggggtggggtggggcggggtcgccgagggc | 71 |
| allele 2 | | | |
| rs2289311, | SNP, T to C, | agggcagcagggtctTgatggccctgcccag | 72 |
| allele 1 | 5′ of exon 27 | | |
| rs2289311, | | agggcagcagggtctCgatggccctgcccag | 73 |
| allele 2 | | | |
| DLGe27, | SNP, C to T, | agatgacaatagcgcCacaaagacgctgtca | 74 |
| allele 1 | within exon 27, | | |
| DLGe27, | synonymous | agatgacaatagcgcTacaaagacgctgtca | 75 |
| allele 2 | | | |
| rs2241831, | SNP, C to A, | ggttactgacagctgCtgagcagtgttcttc | 76 |
| allele 1 | 5′ of exon 29 | | |
| rs2241831, | | ggttactgacagctgAtgagcagtgttcttc | 77 |
| allele 2 | | | |
| rs2241833, | SNP, C to T, | cctggatgcctgggaCgacagacatgacaga | 78 |
| allele 1 | intron | | |
| rs2241833, | | cctggatgcctgggaTgacagacatgacaga | 79 |
| allele 2 | | | |
| rs2579150, | SNP, C to T, | ggctgttttcttagcCgtggagaagcccgcg | 80 |
| allele 1 | 3′ of exon 31 | | |
| rs2579150, | | ggctgttttcttagcTgtggagaagcccgcg | 81 |
| allele 2 | | | |
| rs1058202, | SNP, G to A, | gccgcctgaggggacGccagactcagctctt | 82 |
| allele 1 | 3′ UTR | | |
| rs1058202, | | gccgcctgaggggacAccagactcagctctt | 83 |
| allele 2 | | | |
| rs1058203, | SNP, C to T, | aagtagaagtctgtcCgtctatgaacatgcg | 84 |
| allele 1 | 3′ UTR | | |
| rs1058203, | | aagtagaagtctgtcTgtctatgaacatgcg | 85 |
| allele 2 | | | |
| rs2165046, | SNP, A to G, | tgtctatgaacatgcAggggaaggatccgga | 86 |
| allele 1 | 3′ UTR | | |
| rs2165046, | | tgtctatgaacatgcGggggaaggatccgga | 87 |
| allele 2 | | | |
| rs2165047, | SNP, G to A, | ctctcctggaaggacGtcacaactccaggtg | 88 |
| allele 1 | 3′ UTR | | |
| rs2165047, | | ctctcctggaaggacAtcacaactccaggtg | 89 |
| allele 2 | | | |
| rs2579151, | SNP, C to G, | tctccagaagcttcaCtcacactccactggt | 90 |
| allele 1 | 3′ of gene | | |
| rs2579151, | | tctccagaagcttcaGtcacactccactggt | 91 |
| allele 2 | | | |
| tsc0376484, | SNP, G to A, | agagttagacttcttGaacaaccttttaagg | 92 |
| allele 1 | 3′ of gene | | |
| tsc0376484, | | agagttagacttcttGaacaaccttttaagg | 93 |
| allele 2 | | | |

Table 3 identifies the SNPs and adjacent sequence as well as the particular allelic version. The table includes novel SNPs identified through mutation detection as well as SNPs from public databases. All the public domain SNPs used have an rs- or tsc-number which will identify them uniquely in the genome.

TABLE 4

| | | |
|---|---|---|
| Name | Sequence | SEQ ID NO: |

| | | |
|---|---|---|
| Dlg5ex1F | 5′-CCATGACGGAGGTGGAAGC | 97 |
| Dlg5ex1R | 5′-AGAGGAGCGAGTCCACCGA | 98 |
| Dlg5ex2F | 5′-GACTGATGATCAGCTGGCTTG | 99 |
| Dlg5ex2R | 5′-CGGAAGGATGATCCTGTGAG | 96 |
| Dlg5ex3F | 5′-CCAGGGGAGGATGCAA | 97 |
| Dlg5ex3R | 5′-ACAAGCACACCACTATCAGGG | 98 |
| Dlg5ex4F | 5′-CCTAATCCAGGACCTGGTTC | 99 |
| Dlg5ex4R | 5′-CTTGCACAGGGACAGGACTAG | 100 |
| Dlg5ex5F | 5′-GCTGTATCTACGGGAAGTGTTG | 101 |
| Dlg5ex5R | 5′-GATCACAGATGTGAGCCAACG | 102 |
| Dlg5ex6F | 5′-CCTTGTCATCAGTCTCACCCTC | 103 |
| Dlg5ex6R | 5′-GAGCCACGATTCCCAAGACA | 104 |
| Dlg5ex7F | 5′-ACATCTCGCCACCTCTCTTG | 105 |
| Dlg5ex7R | 5′-TGCTGTAGGAGAGGCTGAAA | 106 |
| Dlg5ex8F | 5′-TCAGCAACCTCTCCCTCTTC | 107 |
| Dlg5ex8R | 5′-ACGCCAGCTTGAGGTCAC | 108 |
| Dlg5ex9F | 5′-TGACCTTGTCCTCTGCTCCT | 109 |
| Dlg5ex9R | 5′-CATTGCCTTGCCCAGAAG | 110 |
| Dlg5ex10F | 5′-CCGTGGCTCTCTCTGTTCAC | 111 |
| Dlg5ex10R | 5′-TCACCGTCTCCTCCTTCATC | 112 |
| Dlg5ex11F | 5′-TTTGTGAAATGGTTGCTGT | 113 |
| Dlg5ex11R | 5′-GTGCTACCTGGCTCTCTTCG | 114 |
| Dlg5ex12F | 5′-CCCCTGAGTGAGAGTTGTGG | 115 |
| Dlg5ex12R | 5′-CCACTGAGGTTGATGTGCAG | 116 |
| Dlg5ex13F | 5′-CACCAGGGTAGATGTAACTGAG | 117 |
| Dlg5ex13R | 5′-GATTCTTATTTCCCTCCCAGAC | 118 |
| Dlg5ex14F | 5′-CTCCTGACTTTGGCACCTTG | 119 |
| Dlg5ex14R | 5′-TTCAGACCAGCGTCCAGTCA | 120 |
| Dlg5ex15aF | 5′-TGCTTTACCTCTGGGGATGG | 121 |
| Dlg5ex15bF | 5′-CTTCCGCTCAGATGCCTCTG | 122 |
| Dlg5ex15cF | 5′-CAGAAGGAGCGACTCCATTAAG | 123 |
| Dlg5ex15aR | 5′-GCAAAGGCACCAGGGTAAAC | 124 |
| Dlg5ex15bR | 5′-GGTAGTAGCTGGAAGCAATGC | 125 |
| Dlg5ex15cR | 5′-CAGAGAGCTTCTCAGGCACTG | 126 |
| Dlg5ex16F | 5′-TGGCCACACTCCACTCTTTC | 127 |
| Dlg5ex16R | 5′-CTCAGGGCTGAAAACACATG | 128 |
| Dlg5ex17F | 5′-GAGCCACAGCCACATTGTGA | 129 |
| Dlg5ex17R | 5′-GGAAGCTTCTCCACCAATGA | 130 |
| Dlg5ex18F | 5′-GAACCCTTGCCTGGTCTGTG | 131 |
| Dlg5ex18R | 5′-GAAAGCAATGGCTCTGACAG | 132 |
| Dlg5ex19F | 5′-GACTGGTAGCCTGGTGGAGA | 133 |
| Dlg5ex19R | 5′-GAAGTTCTCAGCTAAGCCCAG | 134 |
| Dlg5ex20F | 5′-GCAATGCAGAGCCTAGCATC | 135 |
| Dlg5ex20R | 5′-TGCTGGGACACTCAAGCTAC | 136 |
| Dlg5ex21F | 5′-CTGCACTGTCAGATCATATGC | 137 |
| Dlg5ex21R | 5′-ACACCAGGATGGGCTCAGTG | 138 |
| Dlg5ex22F | 5′-GAACAGCAGTGCTCCCAAGT | 139 |
| Dlg5ex22R | 5′-CCAGAACTTACGGCTGGCAC | 140 |
| Dlg5ex23F | 5′-GCTCAGATCTAGTTGCCACAGG | 141 |
| Dlg5ex23R | 5′-CCACTTGGAGAATGTGCTCAG | 142 |
| Dlg5ex24F | 5′-CCAAGAAGCAGGCAGAAAGC | 143 |
| Dlg5ex24R | 5′-TGTACTCCTCCGTCTTTGGTG | 144 |
| Dlg5ex25F | 5′-GACTCAGTCCTTCCTGCAGAG | 145 |
| Dlg5ex25R | 5′-CACCAGGAAAAGAGTCTCCAG | 146 |
| Dlg5ex26F | 5′-CTCGGCGATTCCTGATCAAG | 147 |
| Dlg5ex26R | 5′-GAAGCAGAATCCCTCCTCCAG | 148 |
| Dlg5ex27F | 5′-GTAGCCTTGAGACCTGCCAAG | 149 |
| Dlg5ex27R | 5′-TGTGGCTGTGAAGATGGCAG | 150 |
| Dlg5ex28F | 5′-GTGCTCATGCTGGACTCCAG | 151 |
| Dlg5ex28R | 5′-CAGGCTTCTGGAACACTGTG | 152 |
| Dlg5ex29F | 5′-GTCAGATTCATGCATGGCAG | 153 |
| Dlg5ex29R | 5′-CAGGCACAGGTGAACTCAGAC | 154 |
| Dlg5ex30F | 5′-CTGTGTGGCTTTACTGCCTTG | 155 |
| Dlg5ex30R | 5′-CCATAGGCCCATCTCTCATTC | 156 |
| Dlg5ex31F | 5′-GCTGTTGCTGTGCTTTATGTG | 157 |
| Dlg5ex31R | 5′-AGAATCCTGACGTTGGCCAG | 158 |
| Dlg5ex32F | 5′-CTGGTGAAGGAGAGTCAGGTG | 159 |
| Dlg5ex32R | 5′-GTGCTTCTGGGTCCTGGTTC | 160 |

Table 4. Primers used for mutation detection of the dlg5 gene. Each primer is named after exon number and either F for forward or R for reverse primer.

TABLE 5

| | | | | | | IBD | | |
|---|---|---|---|---|---|---|---|---|
| | IBD families and trios | | | | | trios | | |
| SNP | T:U | T/U | chi 2 | p-value | T:U | T/U | chi 2 | p-value |
| TSC0376484 | 272:198 | 1.37 | 11.65 | p = 0.0006 | 206:150 | 1.37 | 8.81 | p = 0.0029 |
| rs2579151 | 275:225 | 1.20 | 5.00 | p = 0.02 | 212:170 | 1.24 | 4.62 | p = 0.03 |
| rs2165047 | 283:232 | 1.21 | 5.05 | p = 0.02 | 214:168 | 1.27 | 5.54 | p = 0.018 |
| rs2165046 | 369:296 | 1.25 | 8.01 | p = 0.004 | 277:212 | 1.30 | 8.64 | p = 0.003 |
| rs1058203 | 286:244 | 1.17 | n.s. | n.s. | 221:181 | 1.22 | 3.98 | p = 0.04 |
| rs2579150 | | | | | 220:178 | 1.23 | 4.43 | p = 0.03 |
| rs2241833 | 287:237 | 1.21 | 4.77 | p = 0.02 | 219:172 | 1.27 | 5.65 | p = 0.017 |
| rs2289311 | 360:287 | 1.25 | 9.22 | p = 0.002 | 267:204 | 1.30 | 8.43 | p = 0.003 |
| rs1058198 | 348:270 | 1.28 | 9.84 | p = 0.0017 | 262:193 | 1.35 | 10.46 | p = 0.001 |
| rs1261990 | 284:236 | 1.20 | 4.43 | p = 0.03 | 221:174 | 1.28 | 5.59 | p = 0.01 |
| DLGe18 | 359:280 | 1.28 | 9.77 | p = 0.002 | 272:206 | 1.32 | 9.11 | p = 0.003 |
| rs2289308 | 349:276 | 1.26 | 8.53 | p = 0.003 | 265:205 | 1.29 | 7.66 | p = 0.005 |
| rs1248634 | 287:235 | 1.22 | 5.18 | p = 0.02 | 220:175 | 1.25 | 5.13 | p = 0.02 |
| rs1248680 | 284:244 | 1.16 | n.s. | n.s. | 217:177 | 1.22 | 4.06 | p = 0.04 |
| rs1248696 | 134:105 | 1.27 | 3.52 | p = 0.06 | 107:84 | 1.27 | 2.9 | p = 0.09 |
| DLGe2 | 128:44 | 2.90 | 41.02 | p = 0.000000 | 102:32 | 3.0 | 34.00 | p = 0.000000 |
| TSC0068513 | 327:281 | 1.16 | n.s. | n.s. | 257:203 | 1.26 | 6.34 | p = 0.011 |

Table 5. Data demonstrating the association between IBD and dlg5.

### EXAMPLE 3

Expression Analysis of DLG5 on a Panel of Normal Tissues and Colon Biopsies from Patients with IBD using Real Time PCR

Real-time PCR-experiments where performed on Applied Biosystems 7900 HT in 384 format with Syber Green chemistry (double-stranded DNA binding dye, minor groove binding) and fluorescent probes. In order to be able to detect any unspecific amplification and melting, curve analysis was performed after each completed PCR.

Each sample was run in duplicate with 4 ng of template in each reaction (10 μl). The PCR reactions were run at 50 cycles for both sample and reverse transcriptase negative controls. Non-template controls where also studied to confirm that the signals were not due to the primers themselves. The primers where designed from cDNA sequences in the program Primer Express, manufactured by Applied Biosystems. The primers were all complementary to the intron exon junction. As internal standard h36b4 (acidic ribosomal phosphoprotein P0) and β-actin were used to normalize for differences in RNA input and cDNA synthesis. The relative expression in the different samples was calculated by the parameter C_{T} (threshold cycle). C_{T} is defined as the fractional cycle number at which the fluorescence passes a threshold above baseline.

The relative expression of the dlg5 gene is calculated with the formula: 2^{-ΔC}_{T} where ΔC_{T} is defined as: (C_{T} DLG5−C_{T}36B4) alternatively (C_{T}DLG5−C_{T}β-actin)
C_{T} DLG5=the threshold cycle for the dlg5 gene in the tissue of interest.
C_{T} 36B4 =the threshold cycle for 36b4 in the same tissue.
C_{T} β-actin=the threshold cycle for β-actin in the same tissue

The primers used for dlg5 (exon27-exon28) oriented in 5′ to 3′ direction were forward: CCAGTGACTCCATTCCACTCTTT (SEQ ID NO: 161) and reverse: CGGTGCAGTCCACCTTCTG(SEQ ID NO: 162). Also primers were used for exon 29-30 forward: GGAGAAGCGGCCATTTCG (SEQ ID NO: 163) and reverse: CTCAATAGCGTGCGGAGCAA(SEQ ID NO: 164), exon 32 forward: CAGTGTGGGTGTCTTCGTTTGG (SEQ ID NO: 165) and reverse: ATTAACAGGACGCATAGCTTAAGGA (SEQ ID NO: 166) on a subset of the material. For primers against exon 29-30 and exon 32, fluorescent probes were used for detection using sequences: exon 29-30: AAAGGAGATCACAGAAAAGAACCGACACTGC (SEQ ID NO: 167) and exon 32: TGAGGCTAGATATGTCTGGCTGAAGATTTGATGTG (SEQ ID NO: 168). Detection of amplified fragments for exon 27-28 was done using Syber green Chemistry.

For endogenous control genes such as acidic ribosomal phosphoprotein P0(h36b4) and β-actin, primer sequences were, forward: CCATTCTATCATCAACGGGTACAA (SEQ ID NO: 169) and reverse: AGCAAGTGGGAAGGTGTAATCC (SEQ ID NO: 170) for h36b4 and forward: AGCCTCGCCTTTGCCGA (SEQ ID NO: 171) and reverse: CTGGTGCCTGGGGCG (SEQ ID NO: 172) for β-actin. For β-actin, detection with a fluorescent probe was used with the sequence CCGCCGCCCGTCCACACCCGCC (SEQ ID NO: 173), while Syber Green Chemistry was used for detection of h36b4.

Tissue Panel

Complementary DNA (cDNA) was purchased from Clontech Laboratories Inc. Twelve different tissues was used: heart, placenta, liver, skeletal muscle, kidney, pancreas as part of human MTCTTM I #K1420-1. Testis, prostate, small intestine as part of human MTCTTM II K#1421-1. Ileum cDNA is a part of human foetal MTCTTM II K#1425-1. Descending Colon is part of human Immune MTCTTM II K#1425-1. Brain mRNA (Human brain, whole #6516-1) from which cDNA was prepared using SuperScript™ First-Strand Synthesis System for RT-PCR (Gibco BRL), was used for the cDNA synthesis. Five hundred nanogram of total RNA was used for each reaction using the oligo dT primer provided in the kit for the reverse transcriptase (RT) reaction and RT negative controls.

Colon Biopsies

Colon biopsies were taken from patients using endoscope and a grasp biopsy tool. Biopsies were taken from the mucosa at different parts of the colon and terminal ileum.

Results

Tissue distribution of DLG confirmed high expression in placenta and testis and also high expression in brain, prostate, descending colon and ileum as shown in Table 6. Analysis of colon biopsies showed a markedly decreased expression in Crohn's (CD) patients compared to non-IBD disease controls. Almost 6-fold difference was seen in one subset of the samples consisting of 6 non-IBD controls and 29 CD cases shown in Table 7. A more modest decrease was seen in an another subset consisting of 16 hospitalised normals(HN)(approx.1.5-fold) and 39 non-IBD controls (DC)(approx. 2-fold) compared to 23 healthy controls as shown in Table 8. Whereas a consistent difference was seen in a third and a fourth subset of 40 ulcerative colitis (UC) and 49 CD samples compared to 25 healthy normals. The results are shown in Table 9 and 10, suggesting a 1.5- and 2.3-fold decrease respectively.

In Summary:

We have found DLG5 to be expressed in most tissues examined: placenta, heart, prostate, skeletal muscle, liver, pancreas, kidney, brain, colon, testis, ileum and small intestine. The highest amount of expression was detected in placenta and ileum. We have also identified a significant difference of expression between colon biopsies from IBD patients relative to non-IBD controls and healthy controls.

TABLE 6
Relative expression of DLG5 in selected human tissues.

| Tissue | 2^{-□Ct} * 1000 |
|---|---|
| Prostate | 7.21 |
| Placenta | 31.1 |
| Heart | 1.7 |
| Skeletal muscle | 1.71 |
| Liver | 0.42 |
| Pancreas | 1.44 |
| Kidney | 2.39 |
| Brain | 19.86 |
| Descending Colon | 5.28 |
| Testis | 6.91 |
| Ileum | 7.9 |
| Small intestine | 2.26 |

TABLE 7
Relative expression analysis using real
time PCR according to formula 2^{-ΔCt} * 1000
using h36b4 as endogenous control.

| Exon 27-28 | DC(n = 6) | CD(n = 29) |
|---|---|---|
| Mean | 23.88 | 4.08 |
| Median | 24.22 | 4.043 |
| Pvalue | 2.82406E−06 | |

Colon biopsies were taken from non IBD disease controls (n = 6) and Crohn's disease patients (n = 29). P-values are calculated using Student's T-test assuming non-equal variance.

TABLE 8
Relative expression analysis using real time PCR
according to formula 2^{-ΔCt} * 1000
for colon biopsies taken from healthy normals,
HN = hospitalised normals (n = 16),
DC = Non-IBD disease controls (n = 40).

| Exon 29-30 | Normal (n = 23) | HN (n = 16) | DC (n = 39) |
|---|---|---|---|
| Mean | 2.778 | 1.939 | 1.476 |
| Median | 2.800 | 1.980 | 1.300 |
| P value | | 4.364E−04 | 5.303E−09 |
| Exon 32 | Normal (n = 25) | HN (n = 16) | DC (n = 39) |
| Mean | 0.954 | 0.630 | 0.420 |
| Median | 0.866 | 0.485 | 0.251 |
| P value | | 3.24E−02 | 1.63E−05 |

P-values are calculated using Student's T-test assuming non-equal variance. Primers against both exon 29-30 and exon 32 were used showing reproducible results.

TABLE 9
Relative expression analysis using real time PCR
according to formula 2^{-ΔCt} * 1000 for colon
biopsies taken from healthy normals and Ulcerative
colitis (UC) patients.

| Exon 29-30 | Normal (n = 25) | UC (n = 40) |
|---|---|---|
| Mean | 2.382 | 1.444 |
| Median | 2.408 | 1.355 |
| P value | 4.31E−07 | |
| Exon 32 | Normal (n = 23) | UC (n = 39) |
| Mean | 1.00 | 0.45 |
| Median | 0.88 | 0.43 |
| P value | 1.10E−06 | |

P-values are calculated using Student's T-test assuming non-equal variance. Primers against both exon 29-30 and exon 32 were used showing reproducible results.

TABLE 10
Relative expression analysis using real time PCR
according to formula 2^{-ΔCt} * 1000 for colon
biopsies taken from health normals and Crohn's disease
(CD) patients.

| Exon 32 | Normal (n = 25) | CD (n = 49) |
|---|---|---|
| Mean | 0.927 | 0.570 |
| Median | 0.881 | 0.517 |
| P value | 1.003E−04 | |
| Exon 29-30 | Normal (n = 24) | CD (n = 49) |
| Mean | 2.508 | 1.568 |
| Median | 2.170 | 1.480 |
| P value | 2.07E−04 | |

P-values are calculated using Student's T-test assuming non-equal variance. Primers against both exon 29-30 and exon 32 were used showing reproducible results.

### EXAMPLE 4

siRNA Inhibition of dlg5 Expression Induces Apoptosis in HeLa Cells

In order to simulate reduced levels or loss of function of dlg5 in a standardized in vitro model of epithelial cells, dlg5 mRNA was knocked down in Hela cells by small interfering RNAs (siRNAs).

For transfection experiments, Hela cells (ATCC) (<passage 20) were plated in 6-well plates at 2-4×10⁵ cells/well, and 24 hrs later transfected with custom-made dlg5 siRNAs or scrambled control siRNA using the TransMessenger transfection kit (all from Qiagen, Hilden, Germany). Cells transfected with the siRNA directed against dlg5 and/or a vector encoding enhanced green fluorescent protein (pEGFP) were analysed 48 hours after transfections. In all experiments, a scrambled, nonspecific siRNA (control siRNA) as well as the transfection reagent TransMessenger (TransM.) alone were used as internal controls. Optimal knockdown of dlg5 transcripts was achieved using 2×10⁵ cells/well and 4 μg/well of the siRNA 5′-GAAGGATGACGTGGACATGCT-3′ (positioned at base 389 of the coding sequence of DLG5—SEQ ED NO: 174), 8 μl/well of Enhancer R and 40 μl/well of TransMessenger reagent. For visualization of transfection efficiency, cells were co-transfected with 2 μg pEGFP-C1 (BD Clontech, Palo Alto, Calif.), seeded on coverslips, fixed for DAPI staining and detection of fluorescence using an Axiophot microscope (Zeiss, Germany), and pictures were captured by a digital camera system (Axiocam, Zeiss).

Expression analyses For estimation of level of knockdown of the dlg5 transcript, mRNA levels of dlg5 and β-actin were analysed by RT-PCR. The following primer pairs were used:

| | |
|---|---|
| β-actin: | |
| 5′-GATGGTGGGCATGGGTCAG-3′ | (SEQ ID NO: 175) |
| and | |
| 5′-CTTAATGTCACGCACGATTTCC-3′, | (SEQ ID NO: 176) |
| dlg5: | |
| 5′-AAACTGTATGACACGGCCATGG-3′ | (SEQ ID NO: 177) |
| and | |
| 5′-CTCCTCCCTGTATTTCTCCGACTC-3′. | (SEQ ID NO: 178) |

In addition, expression of the interferon inducible gene, OAS1 was measured in order to exclude signalling artefacts which might be induced by siRNA. Primers used for detection of OAS1 were 5′-ACCATGCCATTGACATCATCTG-3′(SEQ ID NO: 179) and 5′-AAGACAACCAGGTCAGCGTCAG-3′ (SEQ ID NO: 180).

Western Blot Analyses

In order to measure apoptosis in DLG5 depleted cells, western blotting analysis of caspase-3 and PARP-1 cleavage was performed. Cell extracts (standardized to 10 μg of total protein/lane) were separated by 12 or 15% denaturing SDS-PAGE and transferred to a polyvinylidene difluoride membrane (Hybond-P; 0.8 mA/cm² for 60 min; Amersham Pharmacia Biotech) by semidry blotting using an electroblotter (Bio-Rad). Antibodies were diluted in blocking buffer. Membranes were subsequently washed, incubated with ECL-Plus Detection Reagent, and exposed to Hyperfilm ECL (both from Amersham Pharmacia Biotech). Primary antibodies directed against caspase-3 and PARP-1 were obtained from Cell Signaling Technology (Beverly, Mass.). Protein contents were normalized by subsequent hybridization with an antibody against β-actin (Sigma-Aldrich, St. Louis, Mo.). Between the stainings with specific Abs, blots were stripped in 2% SDS, 62.5 mM Tris, and 100 mM 2-ME for 30 min at 50° C., washed, and blocked again. The bands were quantified using the densitometry program SigmaGel (Jandel Scientific, San Rafael, Calif.). All Western blots were exposed to film for varying lengths of time, and only films generating subsaturating levels of intensity were selected for densitometrical and statistical evaluation. Linearity was assured in independent experiments by using different amounts of material and multiple film exposures (data not shown). Each Western blotting experiment was conducted with two separate membranes in parallel to detect potential stripping artifacts.

Results

siRNA inhibition of dlg5 expression strongly induced apoptosis of Hela cells without any further pro-apoptotic stimuli. Apoptosis was determined by immunoblots detecting activation of the key apoptotic effector caspase-3 and cleavage of its substrate poly(ADP-ribose)polymerase-1 (PARP-1). Moreover, co-transfection of a vector encoding enhanced green fluorescent protein (pEGFP) revealed that after 48 hours, up to 50% of the siRNA-transfected Hela cells showed fragmented nuclei after DAPI staining, which is a hallmark of apoptosis (FIG. 1). The effect was not due to an interferon response (Bridge, A. J. et al. Nat Genet 34,263-264 (2003), since transcript levels of 2′5′-oligoadenylate synthetase (OAS1) were not influenced by dlg5 siRNA (data not shown). Initial time kinetics revealed that DLG5 mRNA levels were significantly reduced 24 hours after siRNA transfection, but even stronger after 48 hours. The extent of apoptosis (determined by cleavage of caspase-3 and PARP-1) closely matched dlg5 mRNA levels. Furthermore, staining of mucosal sections from homozygous CD patients carrying the disease associated variant of rs2289310, ggcctagcaccccccAagccaagcagagcag (SEQ ID NO: 181) (n=3) for cleaved caspase-3 as a marker for apoptosis showed increased apoptotic staining compared to that for patients homozygous for the wildtype allele, ggcctagcaccccccCagccaagcagagcag (SEQ ID NO: 182) (n-3).

In Summary

These novel findings suggest that functional DLG5 is an essential factor for epithelial cell survival.

### EXAMPLE 5

Expression Analyses of DLG5 on Colon Samples from Mice Treated with Dextran Sulphate Sodium (DSS).

The DSS mouse colitis model was used to investigate the expression pattern of DLG5 during acute colitis as well as during the recovery phase of DSS induced colitis.

Mice

7-12 weeks old C57BL/6 mice were exposed to DSS (Dextran sulphate sodium; TDB Consultancy AB, Sweden) added to the drinking water at a concentration of 3% for 6 days. Animals in the recovery phase received DSS for 5 days followed by a water period of 2 weeks (d5+14). C57BL/6 mice not exposed to DSS were used as control animals. For each group five individual animals were analysed. After 7 days (acute phase) or after d5+14 (recovery phase) animals were sacrificed and tissue samples from spleen, large intestine and small intestine were collected. Tissues were flushed in NaCl and then quick-frozen in liquid nitrogen before RNA preparations.

RNA Preparations

RNA from approximately 50 mg of tissue from spleen, large intestine and small intestine was prepared using the TRIZOL method (Invitrogen) according to the manufacturer's instructions. After DNase treatment, cDNA synthesis was performed using Superscript First Strand Synthesis Kit for RT-PCR (Invitrogen Life Technologies).

Real Time PCR

Real-time PCR-experiments where performed on Applied Biosystems 7900 HT in 384 format with Syber Green chemistry (double-stranded DNA binding dye, minor groove binding) and fluorescent probes. In order to detect any unspecific amplification and melting, curve analysis was performed after each completed PCR.

Each sample was run in triplicate using 3 ng of template for each reaction (10 μl). The PCR reactions were run at 40 cycles for both sample and reverse transcriptase negative controls. Non-template controls where also included to confirm that the signals were not due to the primers themselves. The primers where designed from cDNA sequences in the program Primer Express, manufactured by Applied Biosystems. The primers were all complementary to the intron-exon junction. Acidic ribosomal phosphoprotein P0 (m36b4) was used to normalize for differences in RNA input and cDNA synthesis.

The relative expression in each tissue was calculated using the formula ΔC_{T}=C_{T} DLG5−C_{T} 36B4 where C_{T} is defined as the fractional cycle number at which the fluorescence passes a threshold above baseline. Relative values for dlg5 in distinct tissues was calculated using the comparative (ΔΔCT) method, where the ΔCT value from spleen was set as reference (User Bulletin#2, ABI PRISM 7700 Sequence Detection System).
C_{T} dlg5=the threshold cycle for the dlg5 gene in the tissue of interest.
C_{T} 36B4=the threshold cycle for 36b4 in the same tissue.
ΔC_{T}=C_{T} DLG5−C_{T} 36B4
ΔΔCT=ΔCT(tissue of interest)−ΔCT(spleen)

The primers used for the analyses of murine dlg5 (exon 29-30) were forward: CAGAAAAGAACCGGCACTGTCT (SEQ ID NO: 183), reverse: TGTGGTGCAGCCTCTCGAT (SEQ ID NO: 184) and probe sequence: CTGGACATCGCCCCGCATGC (SEQ ID NO: 185)

As endogenous control, the mouse gene for acidic ribosomal phosphoprotein P0(m36b4) was analysed using following primer sequences, forward: GAG GAA TCA GAT GAG GAT ATG GGA (SEQ ID NO: 186) and reverse: AAG CAG GCT GAC TTG GTT GC (SEQ ID NO: 187). For detection of m36b4 a fluorescent probe with the sequence TCG GTC TCT TCG ACT AAT CCC GCC AA (SEQ ID NO: 188) was used.

Results

The expression levels of dlg5 was analysed in samples from large (colon) and small intestine (terminal ileum) from C57BL/6 mice in acute and recovery phase of DSS induced colitis. Interestingly, only samples from large intestine showed significant variations between different phases of disease progression (FIG. 2). During the acute phase of colitis, the expression level of dlg5 in large intestine was found to be significantly lower than that found in corresponding samples from control animals or animals in recovery phase. Also, in large intestine an increased expression level during the recovery phase was detected. No significant differences in dlg5 expression levels could be detected when samples from small intestine during different phases of disease progression were analysed. As the intestinal inflammation is limited to the large intestine in this model, the expression pattern suggests a link to the colon and/or inflammation.

In Summary

We have found variations in the expression of dlg5 in large intestine during distinct phases of colitis in mice. These results, from an animal model, strengthen the role for DLG5 during colitis disease progression.

1. A method for identifying a compound capable of modulating the action of the DLG5 protein which method comprises subjecting one or more test compounds to a screen comprising a polypeptide containing the amino acid sequence shown in SEQ ID NO: 2, or a homologue thereof or a fragment of either.

2. A method for identifying potential anti-IBD therapeutic compounds comprising contacting an assay system capable of detecting the effect of a test compound against expression level of DLG5, with a test compound and assessing the change in expression level of DLG5.

3. A method of screening for a compound potentially useful for treatment of IBD, which comprises assaying the compound for its ability to modulate the activity or amount of DLG5.

4. The method according to claim 3, which involves a method selected from:
(i) measurement of DLG5 activity using a cell line which expresses the DLG5 polypeptide or using purified DLG5 polypeptide; and
(ii) measurement of dlg5 transcription or translation in a cell line expressing the DLG5 polypeptide.

5. A cell comprising a reporter gene under the control of the dlg5 promoter.

6. A method of testing potential therapeutic agents for the ability to suppress IBD phenotype comprising contacting a test compound with a cell engineered to express the DLG5 polypeptide; and determining whether said test compound modulates expression of the DLG5 polypeptide.

7. A method for identifying inhibitors of transcription of dlg5, which method comprises contacting a potential therapeutic agent with a cell or cell line as described above and determining inhibition of dlg5 transcription by the potential therapeutic agent by reference to a lack of or reduction in expression of the reporter gene.

8. A method of preparing a pharmaceutical composition comprising:
i) identifying a compound as useful for treatment of IBD according to a screening method as described herein; and
ii) mixing the compound or a pharmaceutically acceptable salt thereof with a pharmaceutically acceptable excipient or diluent.

9. Use of a compound able to modulate the activity or amount of DLG5 in the preparation of a medicament for the treatment of IBD.

10. A method for the diagnosis of IBD or determining susceptibility to develop IBD, which method comprises:
(i) obtaining a protein or nucleic acid containing sample from an individual;
(ii) detecting the presence or absence of a variant DLG5 on the basis of the presence of a polymorphic amino acid within the DLG5 protein, or a polymorphic base within the dlg5 gene sequence; and,
(iii) determining the status of the human by reference to the presence or absence of a polymorphism in DLG5.
